SEQUENCE LISTING (12 sequences)

SEQ ID NO: 1 - protein, 374 aa, Homo sapiens
MVLWESPRQCSSWTLCEGFCWLLLLPVMLLIVARPVKLAAFPTSLSDCQTPTGWNCSGYDDRENDLFLCDTNTCKFDGECLRIGDTVTCVCQFKCNNDYVPVCGSNGESYQNECYLRQAACKQQSEILVVSEGSCATDAGSGSGDGVHEGSGETSQKETSTCDICQFGAECDEDAEDVWCVCNIDCSQTNFNPLCASDGKSYDNACQIKEASCQKQEKIEVMSLGRCQDNTTTTTKSEDGHYARTDYAENANKLEESAREHHIPCPEHYNGFCMHGKCEHSINMQEPSCRCDAGYTGQHCEKKDYSVLYVVPGPVRFQYVLIAAVIGTIQIAVICVVVLCITRKCPRSNRIHRQKQNTGHYSSDNTTRASTRLI

SEQ ID NO: 2 - DNA, 32 bp, Artificial Sequence
  source: /note=“Description of Artificial Sequence Synthetic primer”
ctatcgatctatcgatatggtgctgtgggagt

SEQ ID NO: 3 - DNA, 63 bp, Artificial Sequence
  source: /note=“Description of Artificial Sequence Synthetic primer”
gactctagagtcacttgtcatcgtcgtccttgtagtcggcgcgccactttttttcacagtgtt

SEQ ID NO: 4 - protein, 11 aa, Artificial Sequence
  source: /note=“Description of Artificial Sequence Synthetic peptide”
WRADYKDDDDK

SEQ ID NO: 5 - DNA, 29 bp, Artificial Sequence
  source: /note=“Description of Artificial Sequence Synthetic primer”
ctgggcgcgccactttttttcacagtgtt

SEQ ID NO: 6 - protein, 28 aa, Artificial Sequence
  source: /note=“Description of Artificial Sequence Synthetic peptide”
KYALADASLKMADPNRFRGKDLPVLSGR

SEQ ID NO: 7 - DNA, 32 bp, Artificial Sequence
  source: /note=“Description of Artificial Sequence Synthetic primer”
cgactctagattagattaacctcgtggacgct

SEQ ID NO: 8 - DNA, 31 bp, Artificial Sequence
  source: /note=“Description of Artificial Sequence Synthetic primer”
ctgctcgagtgtgatatttgccagtttggtg

SEQ ID NO: 9 - DNA, 32 bp, Artificial Sequence
  source: /note=“Description of Artificial Sequence Synthetic primer”
ctgctcgagacaccacataccttgtccggaac

SEQ ID NO: 10 - protein, 56 aa, Homo sapiens
MVLWESPRQCSSWTLCEGFCWLLLLPVMLLIVARPVKLAAFPTSLSDCQTPTGWNC

SEQ ID NO: 11 - protein, 6 aa, Artificial Sequence
  source: /note=“Description of Artificial Sequence Synthetic 6xHis tag”
HHHHHH

SEQ ID NO: 12 - protein, 5 aa, Artificial Sequence
  source: /note=“Description of Artificial Sequence Synthetic 5xHis tag”
HHHHH

(19) United States
(12) Patent Application Publication
(10) Pub. No.: US 2011/0159588 A1
(43) Pub. Date: Jun. 30, 2011

(54) Methods for Modulating a PDGF-AA Mediated Biological Response

(76) Inventor: Kui Lin, Foster City, CA (US)

(21) Appl. No.: 12/980,074

(22) Filed: Dec. 28, 2010

Related U.S. Application Data
(60) Provisional application No. 61/291,030, filed on Dec. 30, 2009.

(51) Int. Cl. C12N 5/02

(52) U.S. Cl. 435/375

(57) ABSTRACT

The present invention is directed towards methods and means for modulating PDGF-AA mediated biological responses and is based, at least in part, on examining the association of various proteins with TMEFF2 and identification of PDGF-AA as a major growth factor that interacts specifically with TMEFF2. The invention provides the first evidence that TMEFF2 can function to regulate PDGF signaling, to help illuminate the seemingly conflicting biological roles of TMEFF2 in human cancers.

### CROSS-REFERENCE TO RELATED APPLICATION

This application claims priority under 35 U.S.C. Section 119(e) and the benefit of U.S. Provisional Application Ser. No. 61/291,030 filed Dec. 30, 2009, the contents of which is incorporated herein by reference in its entirety.

### FIELD OF THE INVENTION

The present invention concerns methods and means for modulating PDGF-AA mediated biological responses.

### BACKGROUND OF THE INVENTION

Platelet-derived growth factors (PDGFs) not only play an important role in developmental and physiological processes, but also are directly implicated in human cancer and other proliferative disorders (reviewed in (Heldin and Westermark, 1999) and (Hoch and Soriano, 2003)). The human genome consists of four PDGF ligands, PDGF A-D, and two receptors, PDGFRα and PDGFRβ All PDGFs can form functional disulfide-linked homodimers, while only PDGF A and B have been shown to form functional heterodimers. PDGFRs also function as homo- and hetero-dimers that differ in their affinities to different PDGF dimers (reviewed in (Heldin and Westermark, 1999) and (Hoch and Soriano, 2003)). The α subunit of PDGFR has been shown to bind the PDGF A, B and C chains, whereas the β subunit is believed to bind only the B and D chains. The biological responses induced by the different PDGF ligands depend on the relative numbers of the receptor subunits on a given cell type and the specific PDGF dimers present.

TMEFF2, also known as tomoregulin (Uchida et al., 1999), TPEF (Liang et al., 2000), HPP1 (Young et al., 2001) and TENB2 (Glynne-Jones et al., 2001), encodes a transmembrane protein that contains a single epidermal growth factor (EGF)-like domain and two follistatin-like modules (Horie et al., 2000) (Gery et al., 2002) (Uchida et al., 1999) (Glynne-Jones et al., 2001). The biological function of TMEFF2 remains elusive with conflicting reports from different groups. Soluble forms of TMEFF2 extracellular domain have been reported to weakly stimulate erbB-4/HER4 tyrosine phosphorylation in MKN 28 gastric cancer cells (Uchida et al., 1999), and promote survival of mesencephalic dopaminergic neurons in primary culture (Horie et al., 2000). Supporting a positive role in cell proliferation, elevated TMEFF2 expression has been associated with higher prostate cancer grade and hormone independence by several groups (Glynne-Jones et al., 2001) (Mohler et al., 2002) (Afar et al., 2004). In contrast, others reported down-regulation of TMEFF2 in androgen-independent prostate cancer xenografts, as well as growth inhibition induced by ectopic expression of TMEFF2 in androgen-independent prostate cancer cell lines (Gery et al., 2002). Moreover, the 5′-region of TMEFF2 gene is frequently hypermethylated in some cancers (Liang et al., 2000) (Young et al., 2001) (Shibata et al., 2002) (Sato et al., 2002) (Wynter et al., 2004) (Belshaw et al., 2004) (Takahashi et al., 2004) (Geddert et al., 2004) (Suzuki et al., 2005a) (Suzuki et al., 2005b), suggesting a possible tumor suppressor role of TMEFF2 in these cancers.

Follistatin module-containing proteins have been previously shown to be able to bind and modulate the function of a variety of growth factors including members of the transforming growth factor beta (TGF-β family, PDGFs, and vascular endothelial growth factor (VEGF)) (Kupprion et al., 1998; Patel, 1998; Patthy and Nikolics, 1993; Raines et al., 1992) (Chang et al., 2003; Harms and Chang, 2003).

### BRIEF DESCRIPTION OF THE DRAWINGS

FIG. 1 Expression and purification of recombinant ECD of TMEFF2. (A)

Hydropathy plot of TMEFF2 protein based on the algorithm of Kyte and Doolittle (Kyte and Doolittle, 1982) and the predicted domain structure based on NT sequencing of the recombinant TECD in this study and Horie et al., 2000 (Horie et al., 2000). SP, signal peptide; FS I, follistatin-like domain I; FS II, follistatin-like domain II; EGF, epidermal growth factor-like domain; TM, transmembrane domain; N-Gly, potential sites for N-linked glycosylation; GAG, potential site of glycosaminoglycan attachment. (B) Schematic representation of the recombinant TECD-FLAG and TECD-Fc fusion proteins aligned with the full length TMEFF2 (TMEFF2-FL). (C) Purified TECD-FLAG and TECD-Fc were analyzed by SDS-PAGE under reducing conditions with Coomassie blue staining (D) Amino terminal sequence of TMEFF2 (SEQ ID NO: 10). NT sequencing of the purified TECD-FLAG and TECD-Fc revealed the cleavage site of the signal peptide. The amino acid sequence identified by NT sequencing is underlined. Arrowhead indicates the signal peptide cleavage site.

FIG. 2 Binding of PDGF ligands and other recombinant proteins to immobilized TECD-FLAG (A,B) and binding of TECD-Fc to immobilized PDGF ligands (C,D). (A) Binding of an anti-FLAG mAb and six Fc-tagged ECD of recombinant proteins to the TECD-FLAG coated wells. HRP-conjugated anti-mouse and anti-human Fey were used to detect anti-FLAG mAb and Fc-tagged proteins, respectively. (B) Binding of dimeric PDGF ligands to TECD-FLAG coated wells. PDGF-AA, AB or BB were applied to TECD-FLAG coated wells (solid symbols) or blank wells (open symbols) and detected with biotinylated anti-PDGF-A (for PDGF-AA & AB) or PDGF-B (for PDGF-BB) antibodies followed by streptavidin-HRP. Anti-PDGF pAb coated wells were used as a positive control for PDGF-AA binding (x). (C) TECD-Fc was applied to wells coated with PDGF-AA, AB or BB and detected with HRP-conjugated anti-human Fey. (D) TECD-Fc and other Fc- or His-tagged ECD of various transmembrane proteins were applied to PDGF-AA coated wells and detected with HRP-conjugated anti-human Fey or biotinylated anti-His antibodies followed by streptavidin-HRP, respectively. EGFR, human epithelial growth factor receptor; HER, human EGF-like receptor; TNFR, tumor necrosis factor receptor; PDGFRβ, PDGF receptorβ; mOX40, murine OX40; DLL4, delta-like protein 4; FLRG, Follistatin-related gene; 2×EGF-His6, His6-tagged tandem array of the EGF module of TMEFF2. Error bars represent standard deviations between duplicates. Representative graphs of at least three independent experiments are shown.

FIG. 3 Co-immunoprecipitation of PDGF-AA or PDGF-AB with full-length or intracellular domain-truncated TMEFF2 expressed on the surface of 293 cells. Anti-PDGF-A blots were run under reducing condition, whereas the anti-TMEFF2 blot was run under non-reducing condition. Multiple bands of TMEFF2-FL and TMEFF2-ΔICD were detected by the anti-TMEFF2 mAb due to different degrees of glycosylation and proteoglycan attachment (Glynne-Jones et al., 2001). mAb, monoclonal antibody; pAb, polyclonal antibody; Ig LC, light chain of the pAb used for the immunoprecipitation.

FIG. 4 Interaction of PDGF-AA with gD-tagged deletion mutants of membrane-bound TMEFF2 lacking either FS I or both FS modules. Anti-PDGF-A blots were run under reducing condition, whereas the anti-TMEFF2 blot was run under non-reducing condition. Multiple bands of TMEFF2-FL and TMEFF2-ΔFS I were detected by the anti-TMEFF2 mAb due to different degrees of glycosylation and proteoglycan attachment (Glynne-Jones et al., 2001). mAb, monoclonal antibody; pAb, polyclonal antibody; Ig LC, light chain of the Ab used for the immunoprecipitation.

FIG. 5 TECD-Fc interferes with PDGF-AA stimulated proliferation of NR6 cells. (A) & (C) Dose-dependent stimulation of BrdU incorporation by PDGF-AA and PDGF-AB in NR6 cells. (B) & (D) Effects of increasing concentrations of TECD-Fc (filled bars) or PDGF sRα (open bars) on 10 ng/ml PDGF-AA (B) or PDGF-AB (D) stimulated BrdU incorporation.

FIG. 6 TMEFF2 expression is downregulated in glioma. (A) Affymetrix signal intensity of TMEFF2 expression in prostate cancer vs non-cancerous tissues based on GeneLogic data. Each open circle represents one patient sample. (B) Affymetrix signal intensity of TMEFF2 expression in normal brain vs brain cancer tissues based on GeneLogic data. (C) & (D) Normalized signals of TMEFF2 (C) and PDGF-A (D) mRNA expression in proneural (PN), proliferative (Prolif), or mesenchymal (MES) subtypes of 36 glioma samples. Mean signals for each subtype are shown as insets. * p<0.05; **, p<0.005. (E) & (F) TMEFF2 expression is negatively correlated with PDGF-A expression in 76 MD Anderson (E) and 57 UCSF (F) HGG samples. Each axis represents normalized signals of each gene. r values indicate Pearson correlation coefficients. All expression data were obtained using Affymetrix HG-U133A and HG-U133B GeneChips from probe 223557_s_at for TMEFF2 and 205463_s_at for PDGF-A, respectively.

FIG. 7 Expression vs. methylation status of TMEFF2 in 7 human tumor types with publicly available data on both Agilent expression and Infinium methylation arrays in TCGA. Methylation levels are plotted on the x-axis by averaging the beta values of the two Infinium probes, cg06856528 and cg18221862, and mRNA expression levels obtained on the Agilent chip are plotted on the y-axis.

FIG. 8 Human TMEFF2 mino acid sequence (SEQ ID NO: 1).

FIG. 9 TECD-Fc competes with PDGF sRα for PDGF-AA binding. (A) PDGF-AA, but not AB, BB, CC or DD, binds to TECD-Fc. TECD-Fc was applied to wells coated with recombinant human PDGF-AA, AB, BB, CC or DD and detected with HRP-conjugated anti-human Fcγ. (B) sRα binds to all three recombinant human PDGF: AA, AB and BB. Biotinylated recombinant sRα (sRα-bio) was applied to wells coated with recombinant human PDGF-AA, AB, BB, CC or DD and detected with streptavidin-HRP. (C) 10 ng/ml TECD-Fc was mixed with increasing concentrations of sRα-bio and applied to PDGF-AA coated wells. Binding between sRα and PDGF-AA was detected using streptavidin-HRP.

FIG. 10 FACS analysis of 293 cells expressing the gD-tagged full-length TMEFF2 or deletion mutants lacking either FS I or both FS modules using anti-gD mAb (black) and four mAbs (red, green, orange and blue) recognizing the FS I module of TMEFF2. Biotinylated anti-mouse IgG was used as a secondary reagent followed by streptavidine-PE. Filled purple, no primary antibody control.

FIG. 11 Comparative transcript expression profiles of TMEFF2 in human tissues. The mRNA expression patterns for TMEFF2 across thousands of human cancer (red dots), normal (green dots) and diseased but non-malignant (blue dots) tissue specimens are shown. Signal intensity is expressed as an average difference value (Polakis, 2005). Mean and 95% confidence intervals of signals from normal and abnormal (both malignant and diseased non-malignant) samples of each tissue are indicated by a grey vertical bar and a grey box, respectively.

FIG. 12 TMEFF2 expression is down-regulated in some cancers. (A) Bar-graphs of mean TMEFF2 mRNA expression levels in indicated tissues. Error bars represent standard errors of the mean. (B) Number of tissues analyzed in each category. [N], Normal tissues; [C], Cancer tissues; [M], metastatic tissues, [D], diseased but non-malignant tissues; * p<0.05; ** p<0.005.

FIG. 13 In situ hybridization analysis of TMEFF2 mRNA expression in normal adult brain and cerebellum (A), fetal spinal cord and spinal ganglion (B), non-malignant prostate (C) and prostate cancer tissues collected on tissue microarrays (TMA) (D).

FIG. 14 (A) Correlations between the beta values of two TCGA array methylation probes for TMEFF2 in the tissues analyzed: colon adenocarcinoma (coad), lung adenocarcinoma (luad), lung squamous cell carcinoma (lusc), glioma (gbm), rectal adenocarcinoma (read), ovarian carcinoma (ov), and renal papillary cell carcinoma (kirp). (B) Pairwise correlations among the three expression probes belonging to TMEFF2.

### SUMMARY OF THE INVENTION

The present invention is based, at least in part, on examining the association of various proteins with TMEFF2 and identification of PDGF-AA as a major growth factor that interacts specifically with TMEFF2. The invention is further based on experimental data showing that the extracellular domain of TMEFF2 interferes with PDGF-AA—stimulated fibroblast proliferation in a dose—dependent manner. These data provide the first evidence that TMEFF2 can function to regulate PDGF signaling, and help to shed light on the seemingly conflicting biological roles of TMEFF2 in human cancers.

In one aspect, the invention concerns a method for modulating a PDGF-AA-mediated biological response comprising inhibiting the interaction of TMEFF2 with said PDGF-AA in a cell.

In one embodiment, the cell is a tumor cell.

In another embodiment, the biological response is PDGF-AA-mediated stimulation of proliferation, survival or migration of the cell, such as a tumor cell.

In yet another embodiment, the modulation is inhibition of the proliferation, survival or migration of the cell, such as tumor cell.

In a further embodiment, the tumor is prostate or colorectal cancer.

In a different aspect, the biological response is PDGF-AA-mediated tumor suppression.

In one embodiment, such modulation is enhancement of PDGFF-AA-mediated tumor suppression.

In another embodiment, the tumor is cancer which may, for example, be glioblastoma, colon cancer or a cancer of the gastrointestinal tract.

In yet another embodiment, the cancer is characterized by reduced TMEFF2 expression.

In a further embodiment, the cancer is characterized by hypermethylation of TMEFF2.

In a different embodiment, the inhibition is performed by administration of an agent inhibiting the binding of PDGF-AA to TMEFF2.

In another embodiment, the binds to PDGF-AA or TMEFF2.

In yet another embodiment, the agent is selected from the group consisting of TMEFF2 fragments, TMEFF2 variants, agonist TMEFF2 antibodies, TMEFF2, and peptide and non-peptide mimetics and antagonists of TMEFF2.

In a further embodiment, the agent comprises a TMEFF2 extracellular domain (ECD) sequence, or a variant thereof.

In a still further embodiment, the agent comprises a TMEFF2 EGF-like domain sequence, or a variant thereof.

In an additional embodiment, the agent comprises a TMEFF2 follistatin-like domain sequence, or a variant thereof.

### DETAILED DESCRIPTION OF THE INVENTION

### I. Definitions

When trade names are used herein, applicants intend to independently include the trade name product formulation, the generic drug, and the active pharmaceutical ingredient(s) of the trade name product.

Unless stated otherwise, the following terms and phrases as used herein are intended to have the following meanings:

The terms “transmembrane protein with EGF-like and two follistatin-like domains 2,” “TMEFF2,” “tomoregulin,” “TPEF,” “HPP1,” and “TENB2” are used interchangeably and refer to a native sequence TMEFF2 polypeptide or a variant thereof. The definition specifically includes the native sequence human TMEFF2 polypeptide of SEQ ID NO: 1, and TMEFF2 variants having at least about 80% amino acid sequence identity with a TMEFF2 which is a: (i) full-length native sequence; (ii) a polypeptide sequence lacking the signal peptide; (iii) an extracellular domain, with or without the signal peptide; (iv) or any other fragment of a full-length TMEFF2 polypeptide sequence. Such TMEFF2 polypeptide variants include, for instance, polypeptides wherein one or more amino acid residues are added, or deleted, at the N- or C-terminus of the full-length native amino acid sequence. Ordinarily, a TMEFF2 polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence TMEFF2 polypeptide sequence, a TMEFF2 polypeptide sequence lacking the signal peptide, an extracellular domain of a TMEFF2 polypeptide, with or without the signal peptide, or any other specifically defined fragment of a full-length TMEFF2 polypeptide sequence. Ordinarily, TMEFF2 polypeptide variants are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, TMEFF2 variant polypeptides will have no more than one conservative amino acid substitution as compared to a native TMEFF2 polypeptide sequence, alternatively no more than 2, 3, 4, 5, 6, 7, 8, 9, or 10 conservative amino acid substitution as compared to the native TMEFF2 polypeptide sequence.

“Sequence identity” is defined as the percentage of residues in the amino acid sequence variant that are identical after aligning the sequences and introducing gaps, if necessary, to achieve the maximum percent sequence identity. Methods and computer programs for the alignment are well known in the art. One such computer program is “Align 2,” authored by Genentech, Inc., which was filed with user documentation in the United States Copyright Office, Washington, D.C. 20559, on Dec. 10, 1991.

The term “antagonist” as defined herein is any molecule that partially or fully blocks, inhibits, or neutralizes a biological activity mediated by a native sequence PDGF-AA by inhibiting the interaction of TMEFF2 and PDGF-AA. In certain embodiments such antagonist binds to PDGF-AA or to TMEFF2. According to one embodiment, the antagonist is a polypeptide. According to another embodiment, the antagonists is an anti-PDGF-AA or an anti-TMEFF2 antibody. According to another embodiment, the antagonist is a small molecule antagonist.

The term “agonist” as defined herein is any molecule that mimics a PDGF-AA-mediated biological response to TMEFF2. In certain embodiments, the agonist binds to PDGF-AA or TMEFF2. According to one embodiment, the agonist is a polypeptide. According to a particular embodiment, the agonist is a fragment or variant of a native sequence TMEFF2 (such as the native sequence human TMEFF2 of SEQ ID NO: 1), such as a TMEFF2 extracellular domain or a fragment or variant thereof. In a further embodiment, the agonist is a mimetic, such as a peptidomimetic, of a native sequence TNEFF2 (such as the native sequence TMEFF2 of SEQ ID NO: 1), or a fragment thereof.

The term “small molecule” refers to any molecule with a molecular weight of about 1500 daltons or less, preferably of about 500 daltons or less.

The term “potentiator” refers to any molecule that enhances a biological response mediated by a PDGF-AA. In one embodiment, the biological response is tumor suppression. In another embodiment, the potentiator enhances the concentration of TMEFF2 on a tumor cell. In a further embodiment, the potentiator binds to PDGF-AA or TMEFF2. According to one embodiment, the potentiator is a polypeptide. According to another embodiment, the potentiator is an anti-PDGF-AA or an anti-TMEFF2 antibody. According to another embodiment, the potentiator is a small molecule, which has a molecular weight of 1500 daltons or less, preferably about 500 daltons or less.

“Treating” or “treatment” or “alleviation” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. These terms indicate the therapeutic and prophylactic uses herein are successful if they ameliorate, lessen or decrease the symptoms, complications or other problems associated with a disease or ameliorate, lessen or decrease the chance of onset or frequency of the symptoms, complications or other problems associated with a disease.

A subject or mammal is successfully “treated” for a tumor or cancer, such as a tumor or cancer characterized by the expression of TMEFF2, if, after receiving a therapeutic amount of an agent (e.g. an antagonist or a potentiator) according to the methods of the present invention, the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent the agent can prevent growth and/or kill existing cancer cells, it can be cytostatic and/or cytotoxic. Reduction of these signs or symptoms can also be felt by the patient.

The parameters for assessing successful treatment and improvement in the disease are readily measurable by procedures familiar to a physician. For cancer therapy, efficacy can be measured, for example, by assessing the time to disease progression (TTP) and/or determining the response rate (RR). Metastasis can be determined by staging tests and by bone scan and tests for calcium level and other enzymes to determine spread to the bone. Computer tomography (CT) scans and magnetic resonance imaging (MRI) techniques can also be performed to look for the presence and spread of cancer. Chest X-rays and measurement of liver enzyme levels by known methods are used to look for metastasis to the lungs and liver, respectively.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of the treatment of, alleviating the symptoms of or diagnosis of a cancer refers to any animal classified as a mammal (aka “patient”), including humans, other higher primates, domestic and farm animals, and zoo, sports, or pet animals, such as dogs, cats, cattle, horses, sheep, pigs, goats, rabbits, etc. Preferably, the mammal is human.

The term “bioavailability” refers to the systemic availability (i.e., blood-plasma levels) of a given amount of drug administered to a patient. Bioavailability is an absolute term that indicates measurement of both the time (rate) and total amount (extent) of drug that reaches the general circulation from an administered dosage form.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, gastrointestinal stromal tumor (GIST), breast cancer, pancreatic cancer, glioblastoma, cervical cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinome, as well as head and neck cancer.

Administration “in combination with” one or more further therapeutic agents includes simultaneous (concurrent) and consecutive administration in any order.

The term “prodrug” as used in this application refers to a precursor or derivative form of a pharmaceutically active substance that is less cytotoxic to cancer cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

By “solid phase” or “solid support” is meant a non-aqueous matrix to which an antibody, an antagonist or a polypeptide of the present invention can adhere or attach. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

As used herein, the term “immunoadhesin” designates antibody-like molecules that combine the binding specificity of a heterologous protein (an “adhesin”) with the effector functions of immunoglobulin constant domains. Structurally, the immunoadhesins comprise a fusion of an amino acid sequence with the desired binding specificity that is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin can be obtained from any immunoglobulin, such as IgG-1, IgG-2, IgG-3, or IgG-4 subtypes, IgA (including IgA-1 and IgA-2), IgE, IgD, or IgM.

A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes.

An “effective amount” of a polypeptide, antibody, antagonist or composition as disclosed herein is an amount sufficient to carry out a specifically stated purpose. An “effective amount” can be determined empirically and by known methods relating to the stated purpose.

The term “therapeutically effective amount” refers to an amount of an antibody, polypeptide or antagonist of this invention effective to “treat” a disease or disorder in a mammal (aka patient). In the case of cancer, the therapeutically effective amount of the drug can reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See the definition herein of “treating”. To the extent the drug can prevent growth and/or kill existing cancer cells, it can be cytostatic and/or cytotoxic.

A “growth inhibitory amount” of a polypeptide, antibody, antagonist or composition of this invention is an amount capable of inhibiting the growth of a cell, especially tumor, e.g., cancer cell, either in vitro or in vivo. A “growth inhibitory amount” of a polypeptide, antibody, antagonist or composition of this invention for purposes of inhibiting neoplastic cell growth can be determined empirically and by known methods or by examples provided herein.

A “cytotoxic amount” of a polypeptide, antibody, antagonist or composition of this invention is an amount capable of causing the destruction of a cell, especially tumor, e.g., cancer cell, either in vitro or in vivo. A “cytotoxic amount” of a polypeptide, antibody, antagonist or composition of this invention for purposes of inhibiting neoplastic cell growth can be determined empirically and by methods known in the art.

The term “antibody” herein is used in the broadest sense and specifically covers monoclonal antibodies, polyclonal antibodies, dimers, multimers, multispecific antibodies (e.g., bispecific antibodies), and antibody fragments, so long as they exhibit the desired biological activity (Miller et al (2003) Jour. of Immunology 170:4854-4861). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

“Antibody fragments” comprise a portion of a full length antibody, generally the antigen binding or variable region thereof. Examples of antibody fragments include Fab, Fab′, F(ab′)₂, and Fv fragments; diabodies; linear antibodies; minibodies (Olafsen et al (2004) Protein Eng. Design & Sel. 17(4):315-323), fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al (1975) Nature 256:495, or may be made by recombinant DNA methods (see for example: U.S. Pat. No. 4,816,567; U.S. Pat. No. 5,807,715). The monoclonal antibodies may also be isolated from phage antibody libraries using the techniques described in Clackson et al (1991) Nature, 352:624-628; Marks et al (1991) J. Mol. Biol., 222:581-597; for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape etc) and human constant region sequences.

An “intact antibody” herein is one comprising a VL and VH domains, as well as a light chain constant domain (CL) and heavy chain constant domains, CH1, CH2 and CH3. The constant domains may be native sequence constant domains (e.g., human native sequence constant domains) or amino acid sequence variant thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact immunoglobulin antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called .alpha., .delta., .epsilon., .gamma., and .mu., respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310).

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, (1991) “Annu Rev. Immunol.” 9:457-92. To assess ADCC activity of a molecule of interest, an in vitro ADCC assay, such as that described in U.S. Pat. No. 5,500,362 and U.S. Pat. No. 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al (1998) PROC. NAT. ACAD. SCI. (USA) (USA) 95:652-656.

“Human effector cells” are leukocytes which express one or more constant region receptors (FcRs) and perform effector functions. Preferably, the cells express at least Fc.gamma.RIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g., from blood or PBMCs as described herein.

The terms “Fc receptor” or “FcR” are used to describe a receptor that binds to the Fc constant region of an antibody. The preferred FcR is a native sequence human FcR. Moreover, a preferred FcR is one which binds an IgG antibody (a gamma receptor) and includes receptors of the FcγRI , FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See review M. in Daeron, “Annu Rev. Immunol.” 15:203-234 (1997)). FcRs are reviewed in Ravetch and Kinet, “Annu Rev. Immunol”., 9:457-92 (1991); Capel et al (1994) Immunomethods 4:25-34; and de Haas et al (1995) J. Lab. Clin. Med. 126:330-41. Other FcRs, including those to be identified in the future, are encompassed by the term “FcR” herein. The term also includes the neonatal receptor, FcRn, which is responsible for the transfer of maternal IgGs to the fetus (Guyer et al (1976) J. Immunol., 117:587 and Kim et al (1994) J. Immunol. 24:249).

“Complement dependent cytotoxicity” or “CDC” refers to the ability of a molecule to lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santoro et al J. Immunol. Methods, 202:163 (1996), may be performed.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a .beta.-sheet configuration, connected by three hypervariable regions, which form loops connecting, and in some cases forming part of, the .beta.-sheet structure. The hypervariable regions in each chain are held together in close proximity by the FRs and, with the hypervariable regions from the other chain, contribute to the formation of the antigen-binding site of antibodies (see Kabat et al (1991) Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al supra) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk (1987) J. Mol. Biol., 196:901-917). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (κ), based on the amino acid sequences of their constant domains.

“Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994). Anti-ErbB2 antibody scFv fragments are described in WO 93/16185; U.S. Pat. Nos. 5,571,894; and 5,587,458.

The term “diabodies” refers to small antibody fragments with two antigen-binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al (1993) Proc. Natl. Acad. Sci. USA 90:6444-6448.

“Humanized” forms of non-human (e.g., rodent) antibodies are chimeric antibodies that contain minimal sequence derived from non-human immunoglobulin. Humanization is a method to transfer the murine antigen binding information to a non-immunogenic human antibody acceptor, and has resulted in many therapeutically useful drugs. The method of humanization generally begins by transferring all six murine complementarity determining regions (CDRs) onto a human antibody framework (Jones et al, (1986) Nature 321:522-525). These CDR-grafted antibodies generally do not retain their original affinity for antigen binding, and in fact, affinity is often severely impaired. Besides the CDRs, select non-human antibody framework residues must also be incorporated to maintain proper CDR conformation (Chothia et al (1989) Nature 342:877). The transfer of key mouse framework residues to the human acceptor in order to support the structural conformation of the grafted CDRs has been shown to restore antigen binding and affinity (Riechmann et al (1992) J. Mol. Biol. 224, 487-499; Foote and Winter, (1992) J. Mol. Biol. 224:487-499; Presta et al (1993) J. Immunol. 151, 2623-2632; Werther et al (1996) J. Immunol. Methods 157:4986-4995; and Presta et al (2001) Thromb. Haemost. 85:379-389). For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see U.S. Pat. No. 6,407,213; Jones et al (1986) Nature, 321:522-525; Riechmann et al (1988) Nature 332:323-329; and Presta, (1992) Curr. Op. Struct. Biol., 2:593-596.

A polypeptide, antibody, antagonist or composition of this invention which “induces cell death” is one which causes a viable cell to become nonviable. The cell is one which expresses a STOP-1 polypeptide, preferably a cell that overexpresses a STOP-1 polypeptide as compared to a normal cell of the same tissue type. Preferably, the cell is a cancer cell, e.g., a breast, ovarian, stomach, endometrial, salivary gland, lung, kidney, colon, thyroid, pancreatic or bladder cell. Cell death in vitro can be determined in the absence of complement and immune effector cells to distinguish cell death induced by antibody-dependent cell-mediated cytotoxicity (ADCC) or complement dependent cytotoxicity (CDC). Thus, the assay for cell death can be performed using heat inactivated serum (i.e., in the absence of complement) and in the absence of immune effector cells. To determine whether a polypeptide, antibody, antagonist or composition of this invention is able to induce cell death, loss of membrane integrity as evaluated by uptake of propidium iodide (PI), trypan blue (see Moore et al. Cytotechnology 17:1-11 (1995)). Preferred cell death-inducing polypeptides, antibodies, antagonists or compositions are those which induce PI uptake in the PI uptake assay in BT474 cells.

A “TMEFF2-expressing cell” or a “PDGF-AA-expressing” is a cell which expresses an endogenous or transfected TMEFF2 or PDGF-AA polypeptide either on the cell surface or in a secreted form. A “TMEFF2-expressing cancer” or a “PDGF-AA-expressing cancer” is a cancer comprising cells that have a TMEFF2 or PDGF-AA polypeptide present on the cell surface or that produce and secrete a TMEFF2 or a PDGF-AA polypeptide. In another embodiment, such cancer optionally produces and secretes sufficient levels of TMEFF2 or PDGF-AA polypeptide, such that a polypeptide, antibody, antagonist or composition of this invention can bind thereto and have a therapeutic effect with respect to the cancer. A cancer which “overexpresses” a TMEFF2 or PDGF-AA polypeptide is one which has significantly higher levels of TMEFF2 or PDGF-AA polypeptide at the cell surface thereof, or produces and secretes, compared to a noncancerous cell of the same tissue type. Such overexpression can be caused by gene amplification or by increased transcription or translation. Polypeptide overexpression can be determined in a diagnostic or prognostic assay by evaluating increased levels of the polypeptide present on the surface of a cell, or secreted by the cell (e.g., via an immunohistochemistry assay using anti-TMEFF2 or anti-PDGF-AA antibodies prepared against an isolated TMEFF2 or PDGF-AA polypeptide which can be prepared using recombinant DNA technology from an isolated nucleic acid encoding the TMEFF2 or the PDGF-AA polypeptide; FACS analysis, etc.). Alternatively, or additionally, one can measure levels of TMEFF2 or PDGF-AA polypeptide-encoding nucleic acid or mRNA in the cell, e.g., via fluorescent in situ hybridization using a nucleic acid based probe corresponding to a TMEFF2- or PDGF-AA-encoding nucleic acid or the complement thereof; (FISH; see WO98/45479 published October, 1998), Southern blotting, Northern blotting, or polymerase chain reaction (PCR) techniques, such as real time quantitative PCR(RT-PCR). One can also study STOP-1 polypeptide overexpression by measuring shed antigen in a biological fluid such as serum, e.g., using antibody-based assays (see also, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al., J. Immunol. Methods 132:73-80 (1990)). Aside from the above assays, various in vivo assays are available to the skilled practitioner. For example, one can expose cells within the body of the mammal to an antibody which is optionally labeled with a detectable label, e.g., a radioactive isotope, and binding of the antibody to cells in the mammal can be evaluated, e.g., by external scanning for radioactivity or by analyzing a biopsy taken from a mammal previously exposed to the antibody.

The word “label” when used herein refers to a detectable compound or composition which is conjugated directly or indirectly to the polypeptide, antibody, antagonist or composition so as to generate a “labeled” a polypeptide, antibody, antagonist or composition. The label can be detectable by itself (e.g. radioisotope labels or fluorescent labels) or, in the case of an enzymatic label, can catalyze chemical alteration of a substrate compound or composition which is detectable.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re^{188, Re}¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodepa, carboquone, meturedepa, and uredepa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR®; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE™. vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® (tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

A “growth inhibitory agent” when used herein refers to a compound or composition which inhibits growth of a cell, especially a TMEFF2- or PDGF-AA-expressing cancer cell, either in vitro or in vivo. Thus, the growth inhibitory agent can be one which significantly reduces the percentage of such cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Doxorubicin” is an anthracycline antibiotic. The full chemical name of doxorubicin is (8S-cis)-10-[(3-amino-2,3,6-trideoxy-.alpha.-L-lyxo-hexapyranosyl)oxy]-7,-8,9,10-tetrahydro-6,8,11-trihydroxy-8-(hydroxyacetyl)-1-methoxy-5,12-napht-hacenedione.

The term “package insert” is used to refer to instructions customarily included in commercial packages of therapeutic products, that contain information about the indications, usage, dosage, administration, contraindications and/or warnings concerning the use of such therapeutic products.

“Maytansinoid drug moiety” means the substructure of an antibody-drug conjugate that has the structure of a maytansine compound. Maytansine was first isolated from the African shrub Maytenus serrata (U.S. Pat. No. 3,896,11). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, and Kawai et al. (1984) Chem. Pharm. Bull. 3441-3451), each of which is expressly incorporated by reference.

### II. Detailed Description

TMEFF2 is a protein that contains a single EGF-like domain and two follistatin-like modules. The biological function of TMEFF2 remains elusive with conflicting reports suggesting both a positive and a negative correlation between TMEFF2 expression and human cancers. The present invention provides experimental data showing that the extracellular domain of TMEFF2 interacts preferentially and specifically with PDGF-AA, but not other forms of PDGF ligands. This interaction interferes with PDGF-AA—stimulated fibroblast proliferation in a dose—dependent manner, which cannot be mediated by the EGF-like domain alone but requires the amino terminal region of the extracellular domain containing the follistatin modules. Interestingly, both shedding of the extracellular domains of TMEFF2 (Lin et al., 2003), and a truncated splice variant of TMEFF2 encoding a secreted protein without the EGF-like and the transmembrane domains (Quayle and Sadar, 2006), have been identified in cancer cells, suggesting a possible functional role of the extracellular region containing the follistatin domains in the ECM.

The data presented herein provide the first evidence that TMEFF2 can function to regulate PDGF signaling, therefore suggesting an important role of TMEFF2 in the development and progression of human cancers. In addition, the results disclosed herein show for the first time that TMEFF2's expression is downregulated in glioma and other cancers and this correlated with DNA methylation. Together these data provide mechanistic insights to the seemingly conflicting roles of TMEFF2 in human cancers.

In cancers overexpressing TMEFF2, regardless of the biological function of TMEFF2, TNEFF2 antibodies conjugated to anti-cancer agents, such as small molecule cytotoxic molecules, can be used in the treatment of cancer. Such cancers include, for example, prostate cancer.

Cancers characterized by TMEFF2 expression where TMEFF2 exhibits a tumor suppressor function, TMEFF2 agonists, such as variants, fragments, mimetics of native sequence TMEFF2, are useful cancer therapeutic agents. Such cancers include, without limitation, certain glioblastomas, colon cancers, and cancers of the gastrointestinal tract.

Polyclonal Antibodies

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent can include the polypeptide to which the antibody binds or a fusion protein thereof. It can be useful to conjugate the immunizing agent to a protein known to be immunogenic in the mammal being immunized. Examples of such immunogenic proteins include, but are not limited to, keyhole limpet hemocyanin, serum albumin, bovine thyroglobulin, and soybean trypsin inhibitor. Examples of adjuvants that can be employed include Freund's complete adjuvant and MPL-TDM adjuvant (monophosphoryl Lipid A or synthetic trehalose dicorynomycolate). The immunization protocol can be selected by one skilled in the art without undue experimentation.

Monoclonal Antibodies

Monoclonal antibodies can be prepared, e.g., using hybridoma methods, such as those described by Kohler and Milstein, Nature, 256:495 (1975) or can be made by recombinant DNA methods (U.S. Pat. No. 4,816,567) or can be produced by the methods described herein in the Example section. In a hybridoma method, a mouse, hamster, or other appropriate host animal is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the polypeptide to which the antibody binds or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice (New York: Academic Press, 1986), pp. 59-103. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high-level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. Kozbor, J. Immunol., 133:3001 (1984); Brodeur et al., Monoclonal Antibody Production Techniques and Applications (Marcel Dekker, Inc.: New York, 1987) pp. 51-63.

The culture medium in which the hybridoma cells are cultured can then be assayed for the presence of monoclonal antibodies directed against the TMEFF2 or PDGF-AA polypeptide. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be subcloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains in place of the homologous murine sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using techniques known in the art.

Anti-TMEFF2 monoclonal antibodies are described, for example, in U.S. Publication Nos. 20040096392; 20080044840; 20080160012, the entire disclosures of which are expressly incorporated by reference herein.

Human and Humanized Antibodies

The anti-TMEFF2 and anti-PDGF-AA antibodies can further comprise humanized antibodies or human antibodies. Humanized forms of non-human (e.g., murine) antibodies are chimeric immunoglobulins, immunoglobulin chains, or fragments thereof (such as Fv, Fab, Fab′, F(ab′).sub.2, or other antigen-binding subsequences of antibodies) that contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin, and all or substantially all of the FR regions are those of a human immunoglobulin consensus sequence. The humanized antibody preferably also will comprise at least a portion of an immunoglobulin constant region (Fc), typically that of a human immunoglobulin. Jones et al., Nature, 321: 522-525 (1986); Riechmann et al., Nature 332: 323-329 (1988); Presta, Curr. Op. Struct. Biol., 2:593-596 (1992).

Methods for humanizing non-human antibodies are known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and in the following scientific publications: Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

Human antibodies can also be produced using various techniques known in the art, including phage display libraries. Hoogenboom and Winter, J. Mol. Biol., 227: 381 (1991); Marks et al., J. Mol. Biol., 222: 581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

Bispecific Antibodies

Bispecific antibodies are monoclonal, preferably human or humanized, antibodies that have binding specificities for at least two different antigens. In the present case, one of the binding specificities is for the TMEFF2 or PDGF-AA polypeptide, the other one is for any other antigen, and preferably for a cell-surface protein or receptor or receptor subunit. For example, the cell-surface protein can be a natural killer (NK) cell receptor. Thus, according to one embodiment, a bispecific antibody of this invention can bind TMEFF2 and bind a NK cell and, optionally, activate the NK cell. According to another embodiment, a bispecific antibody of this invention can bind TMEFF2 and binds to a stromal tissue compared to other tissue (e.g., stromal targeting agent). If the target is prostate cancer, the bispecific antibody may bind to TMEFF2 and to another prostate cancer antigen.

Methods for making bispecific antibodies are known in the art. Traditionally, the recombinant production of bispecific antibodies is based on the co-expression of two immunoglobulin heavy-chain/light-chain pairs, where the two heavy chains have different specificities. Milstein and Cuello, Nature, 305: 537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10: 3655-3659 (1991).

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies, see, for example, Suresh et al., Methods in Enzymology, 121: 210 (1986).

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

Heteroconjugate Antibodies

Heteroconjugate antibodies are composed of two covalently joined antibodies. Such antibodies have, for example, been proposed to target immune-system cells to unwanted cells (U.S. Pat. No. 4,676,980), and for treatment of HIV infection. WO 91/00360; WO 92/200373; EP 03089. It is contemplated that the antibodies can be prepared in vitro using known methods in synthetic protein chemistry, including those involving crosslinking agents. For example, immunotoxins can be constructed using a disulfide-exchange reaction or by forming a thioether bond. Examples of suitable reagents for this purpose include iminothiolate and methyl-4-mercaptobutyrimidate and those disclosed, for example, in U.S. Pat. No. 4,676,980 the entire disclosure of which is expressly incorporated by reference herein.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See, Caron et al., J. Exp. Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al., Cancer Research, 53: 2560-2565 (1993). Alternatively, an antibody can be engineered that has dual Fc regions and can thereby have enhanced complement lysis and ADCC capabilities. See, Stevenson et al., Anti-Cancer Drug Design. 3: 219-230 (1989).

Cysteine engineered TMEFF2 antibodies are disclosed, for example, in U.S. Pat. No. 7,521,541 and U.S. Publication No. 20090117100, the entire disclosures of which are expressly incorporated by reference herein.

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radio conjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See, WO94/11026.

In another embodiment, the antibody can be conjugated to a “receptor” (such as streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

TMEFF2 antibody drug conjugates are disclosed in U.S. Publication No. 20090117100, the entire disclosure of which is hereby expressly incorporated by reference.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See, Gabizon et al., J. National Cancer Inst., 81(19): 1484 (1989).

Assaying Inhibition of Cell Proliferation

The inhibitory activity of antagonists of this invention can be measured using assays known in the art.

Animal models of tumors and cancers (e.g., breast cancer, colon cancer, prostate cancer, lung cancer, etc.) include both non-recombinant and recombinant (transgenic) animals. Non-recombinant animal models include, for example, rodent, e.g., murine models. Such models can be generated by introducing tumor cells into syngeneic mice using standard techniques, e.g., subcutaneous injection, tail vein injection, spleen implantation, intraperitoneal implantation, implantation under the renal capsule, or orthopin implantation, e.g., colon cancer cells implanted in colonic tissue. See, e.g., PCT publication No. WO 97/33551, published Sep. 18, 1997. Probably the most often used animal species in oncological studies are immunodeficient mice and, in particular, nude mice. The observation that the nude mouse with thymic hypo/aplasia could successfully act as a host for human tumor xenografts has lead to its widespread use for this purpose. The autosomal recessive nu gene has been introduced into a very large number of distinct congenic strains of nude mouse, including, for example, ASW, A/He, AKR, BALB/c, B10.LP, C17, C3H, C57BL, C57, CBA, DBA, DDD, I/st, NC, NFR, NFS, NFS/N, NZB, NZC, NZW, P, RIII and SJL. In addition, a wide variety of other animals with inherited immunological defects other than the nude mouse have been bred and used as recipients of tumor xenografts. For further details see, e.g., The Nude Mouse in Oncology Research, E. Boven and B. Winograd, eds. (CRC Press, Inc., 1991).

The cells introduced into such animals can be derived from known tumor/cancer cell lines, such as any of the above-listed tumor cell lines, and, for example, the B104-1-1 cell line (stable NIH-3T3 cell line transfected with the neu protooncogene); ras-transfected NIH-3T3 cells; Caco-2 (ATCC HTB-37); or a moderately well-differentiated grade II human colon adenocarcinoma cell line, HT-29 (ATCC HTB-38); or from tumors and cancers. Samples of tumor or cancer cells can be obtained from patients undergoing surgery, using standard conditions involving freezing and storing in liquid nitrogen. Karmali et al., Br. J. Cancer, 48: 689-696 (1983).

Tumor cells can be introduced into animals such as nude mice by a variety of procedures. The subcutaneous (s.c.) space in mice is very suitable for tumor implantation. Tumors can be transplanted s.c. as solid blocks, as needle biopsies by use of a trochar, or as cell suspensions. For solid-block or trochar implantation, tumor tissue fragments of suitable size are introduced into the s.c. space. Cell suspensions are freshly prepared from primary tumors or stable tumor cell lines, and injected subcutaneously. Tumor cells can also be injected as subdermal implants. In this location, the inoculum is deposited between the lower part of the dermal connective tissue and the s.c. tissue.

Tumors that arise in animals can be removed and cultured in vitro. Cells from the in vitro cultures can then be passaged to animals. Such tumors can serve as targets for further testing or drug screening. Alternatively, the tumors resulting from the passage can be isolated and RNA from pre-passage cells and cells isolated after one or more rounds of passage analyzed for differential expression of genes of interest. Such passaging techniques can be performed with any known tumor or cancer cell lines.

For example, Meth A, CMS4, CMS5, CMS21, and WEHI-164 are chemically induced fibrosarcomas of BALB/c female mice (DeLeo et al., J. Exp. Med., 146: 720 (1977)), which provide a highly controllable model system for studying the anti-tumor activities of various agents. Palladino et al., J. Immunol., 138: 4023-4032 (1987). Briefly, tumor cells are propagated in vitro in cell culture. Prior to injection into the animals, the cell lines are washed and suspended in buffer, at a cell density of about 10×10⁶ to 10×10⁷ cells/ml. The animals are then infected subcutaneously with 10 to 100 μl of the cell suspension, allowing one to three weeks for a tumor to appear.

One way of evaluating the efficacy of a test compound in an animal model with an implanted tumor is to measure the size of the tumor before and after treatment. Traditionally, the size of implanted tumors has been measured with a slide caliper in two or three dimensions. The measure limited to two dimensions does not accurately reflect the size of the tumor; therefore, it is usually converted into the corresponding volume by using a mathematical formula. However, the measurement of tumor size is very inaccurate. The therapeutic effects of a drug candidate can be better described as treatment-induced growth delay and specific growth delay. Another important variable in the description of tumor growth is the tumor volume doubling time. Computer programs for the calculation and description of tumor growth are also available, such as the program reported by Rygaard and Spang-Thomsen, Proc. 6th Int. Workshop on Immune-Deficient Animals, Wu and Sheng eds. (Base1, 1989), p. 301. It is noted, however, that necrosis and inflammatory responses following treatment can actually result in an increase in tumor size, at least initially. Therefore, these changes need to be carefully monitored, by a combination of a morphometric method and flow cytometric analysis.

Further, recombinant (transgenic) animal models can be engineered by introducing the coding portion of the TMEFF2 and/or PDGF-AA gene into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g., baboons, chimpanzees and monkeys. Techniques known in the art to introduce a transgene into such animals include pronucleic microinjection (U.S. Pat. No. 4,873,191); retrovirus-mediated gene transfer into germ lines (e.g., Van der Putten et al., Proc. Natl. Acad. Sci. USA, 82: 6148-615 (1985)); gene targeting in embryonic stem cells (Thompson et al., Cell, 56: 313-321 (1989)); electroporation of embryos (Lo, Mol. Cell. Biol., 3: 1803-1814 (1983)); and sperm-mediated gene transfer. Lavitrano et al, Cell, 57: 717-73 (1989). For a review, see for example, U.S. Pat. No. 4,736,866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA, 89: 6232-636 (1992). The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry. The animals are further examined for signs of tumor or cancer development.

Alternatively, “knock-out” animals can be constructed that have a defective or altered gene encoding a TMEFF2 and/or PDGF-AA polypeptide, as a result of homologous recombination between the endogenous gene encoding such polypeptide(s) and altered genomic DNA encoding the same polypeptide introduced into an embryonic cell of the animal. For example, cDNA encoding a particular desired polypeptide can be used to clone genomic DNA encoding that polypeptide in accordance with established techniques. A portion of the genomic DNA encoding a particular desired polypeptide can be deleted or replaced with another gene, such as a gene encoding a selectable marker that can be used to monitor integration. Typically, several kilobases of unaltered flanking DNA (both at the 5′ and 3′ ends) are included in the vector. See, e.g., Thomas and Capecchi, Cell, 51: 503 (1987) for a description of homologous recombination vectors. The vector is introduced into an embryonic stem cell line (e.g., by electroporation) and cells in which the introduced DNA has homologously recombined with the endogenous DNA are selected. See, e.g., Li et al., Cell 69: 915 (1992). The selected cells are then injected into a blastocyst of an animal (e.g., a mouse or rat) to form aggregation chimeras. See, e.g., Bradley, in Teratocarcinomas and Embryonic Stem Cells: A Practical Approach, E. J. Robertson, ed. (IRL: Oxford, 1987), pp. 113-152. A chimeric embryo can then be implanted into a suitable pseudopregnant female foster animal and the embryo brought to term to create a “knock-out” animal. Progeny harboring the homologously recombined DNA in their germ cells can be identified by standard techniques and used to breed animals in which all cells of the animal contain the homologously recombined DNA. Knockout animals can be characterized, for instance, by their ability to defend against certain pathological conditions and by their development of pathological conditions due to absence of the polypeptide.

The efficacy of antibodies specifically binding the TMEFF2 or PDGF-AA polypeptides, and other drug candidates, can be tested also in the treatment of spontaneous animal tumors. A suitable target for such studies is the feline oral squamous cell carcinoma (SCC). Feline oral SCC is a highly invasive, malignant tumor that is the most common oral malignancy of cats, accounting for over 60% of the oral tumors reported in this species. It rarely metastasizes to distant sites, although this low incidence of metastasis can merely be a reflection of the short survival times for cats with this tumor. These tumors are usually not amenable to surgery, primarily because of the anatomy of the feline oral cavity. At present, there is no effective treatment for this tumor. Prior to entry into the study, each cat undergoes complete clinical examination and biopsy, and is scanned by computed tomography (CT). Cats diagnosed with sublingual oral squamous cell tumors are excluded from the study. The tongue can become paralyzed as a result of such tumor, and even if the treatment kills the tumor, the animals may not be able to feed themselves. Each cat is treated repeatedly, over a longer period of time. Photographs of the tumors will be taken daily during the treatment period, and at each subsequent recheck. After treatment, each cat undergoes another CT scan. CT scans and thoracic radiograms are evaluated every 8-weeks thereafter. The data are evaluated for differences in survival, response, and toxicity as compared to control groups. Positive response may require evidence of tumor regression, preferably with improvement of quality of life and/or increased life span.

In addition, other spontaneous animal tumors, such as fibrosarcoma, adenocarcinoma, lymphoma, chondroma, or leiomyosarcoma of dogs, cats, and baboons can also be tested. Of these, mammary adenocarcinoma in dogs and cats is a preferred model as its appearance and behavior are very similar to those in humans. However, the use of this model is limited by the rare occurrence of this type of tumor in animals.

Pharmaceutical Compositions

The therapeutic agents, such as antibodies, herein, can be administered for the treatment of various disorders as noted above and below in the form of pharmaceutical compositions.

Lipofectins or liposomes can be used to deliver the polypeptides, nucleic acid molecules, antibodies, antagonists or composition of this invention into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993).

The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

Methods of Treatment

It is contemplated that the various therapeutic agents, such as antibodies, herein can be used in the treatment of cancer, such as, for example, prostate cancer.

The therapeutic agents, such as antibodies, are administered to a mammal, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Intravenous administration of the antibody is preferred.

Other therapeutic regimens can be combined with the administration of the antibodies of the instant invention as noted above. For example, if the antibodies are to treat cancer, the patient to be treated with such antibodies can also receive radiation therapy. Alternatively, or in addition, a chemotherapeutic agent can be administered to the patient. Preparation and dosing schedules for such chemotherapeutic agents can be used according to manufacturers' instructions or as determined empirically by the skilled practitioner. Preparation and dosing schedules for such chemotherapy are also described in Chemotherapy Service, Ed., M. C. Perry (Williams & Wilkins: Baltimore, Md., 1992). The chemotherapeutic agent can precede, or follow administration of the antibody, or can be given simultaneously therewith. The antibody can be combined with an anti-estrogen compound such as tamoxifen or EVISTA™ or an anti-progesterone such as onapristone (see, EP 616812) in dosages known for such molecules.

If the antibodies are used for treating cancer, or for use in the treatment of cancer or in the preparation of a medicament to treat cancer, they can be, optionally, administered with antibodies against other tumor-associated antigens, such as antibodies that bind to one or more of the ErbB2, EGFR, ErbB3, ErbB4, or VEGF receptor(s). These also include the agents set forth above. Also, the antibody is suitably administered serially or in combination with radiological treatments, whether involving irradiation or administration of radioactive substances. Alternatively, or in addition, two or more antibodies binding the same or two or more different antigens disclosed herein can be co-administered to the patient. In a preferred embodiment, the antibodies herein are co-administered with a growth-inhibitory agent. For example, the growth-inhibitory agent can be administered first, followed by an antibody of the present invention. However, simultaneous administration or administration of the antibody of the present invention first is also contemplated. Suitable dosages for the growth-inhibitory agent are those presently used and can be lowered due to the combined action (synergy) of the growth-inhibitory agent and the antibody herein.

All publications (including patents and patent applications) cited herein are hereby incorporated in their entirety by reference.

Further details of the invention are provided in the following non-limiting examples.

### EXAMPLE

### Materials and Methods

### Cell Culture and Reagents

The 293 and NR6 cells were maintained at 37° C. and 5% CO₂ in DMEM/Ham's F-12 (1:1) containing 10% fetal bovine serum (FBS) or RPMI 1640 containing 10% calf serum, respectively. Recombinant human PDGF-AA, AB, BB, CC and DD, recombinant human PDGF sRα, recombinant human PDGFRβ-Fc, recombinant human NGFR-Fc, recombinant mouse FLRG-His6, goat anti-human PDGF, and biotinylated goat anti-human PDGF-A and PDGF-B antibodies were obtained from R&D Systems (Minneapolis, Minn.); rabbit anti-PDGF-A polyclonal antibody was obtained from Santa Cruz Biotechnology (Santa Cruz, Calif.). Mouse anti-FLAG antibody was obtained from Sigma (St. Louis, Mo.). Other recombinant proteins and antibodies were generated at Genentech, Inc.

Generation of the Various Deletion and Fusion TMEFF2 Constructs

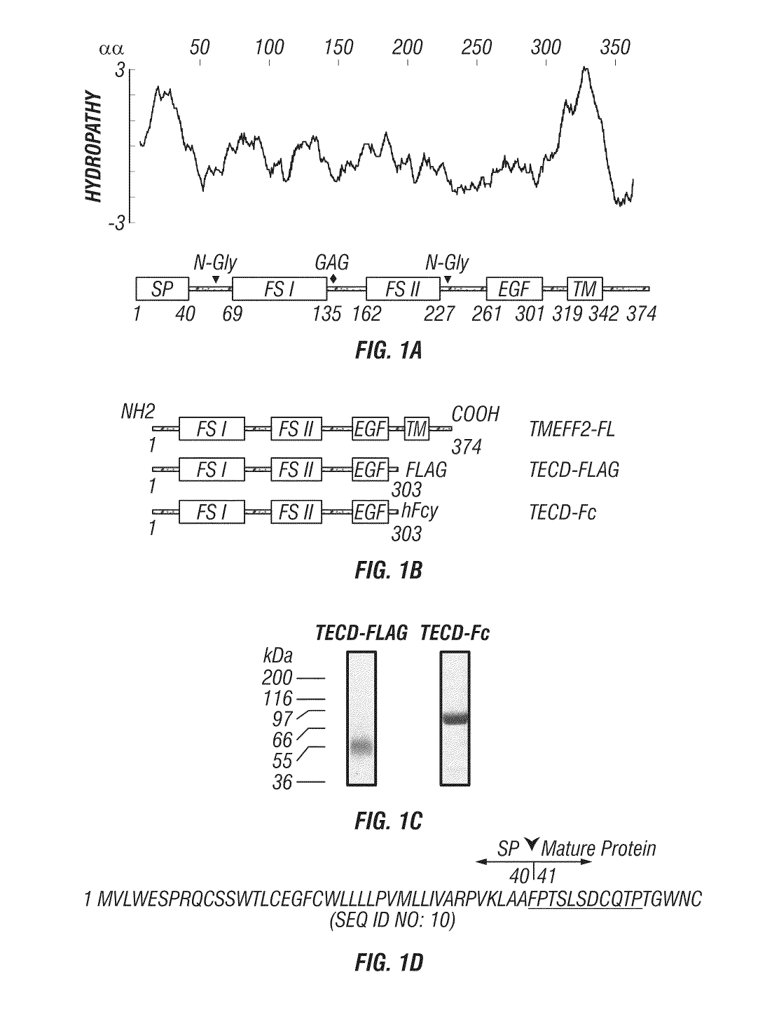

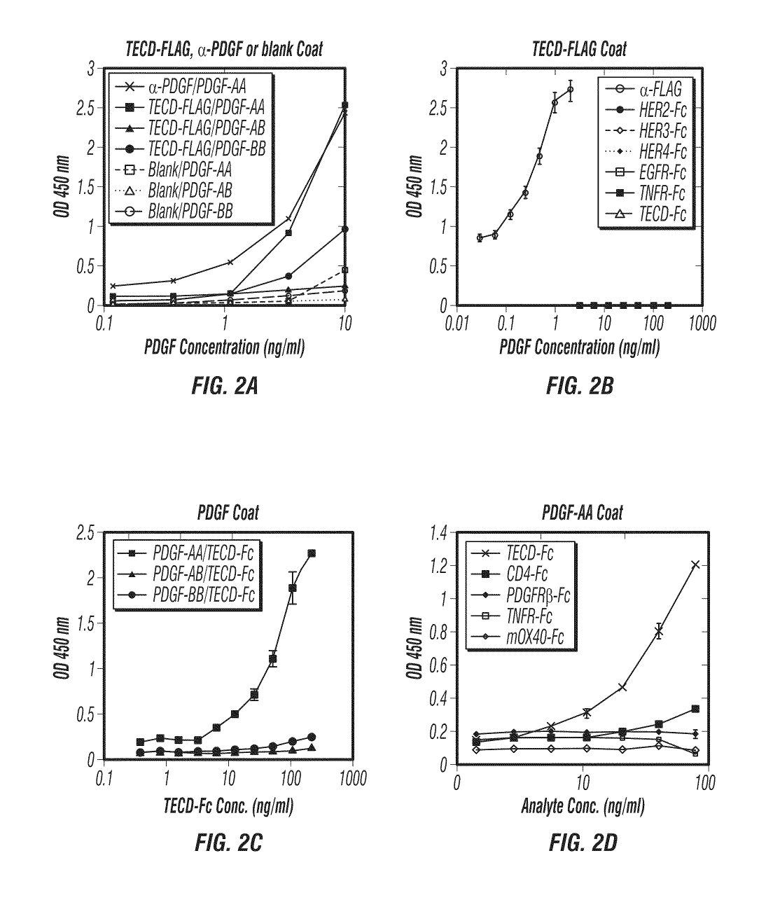

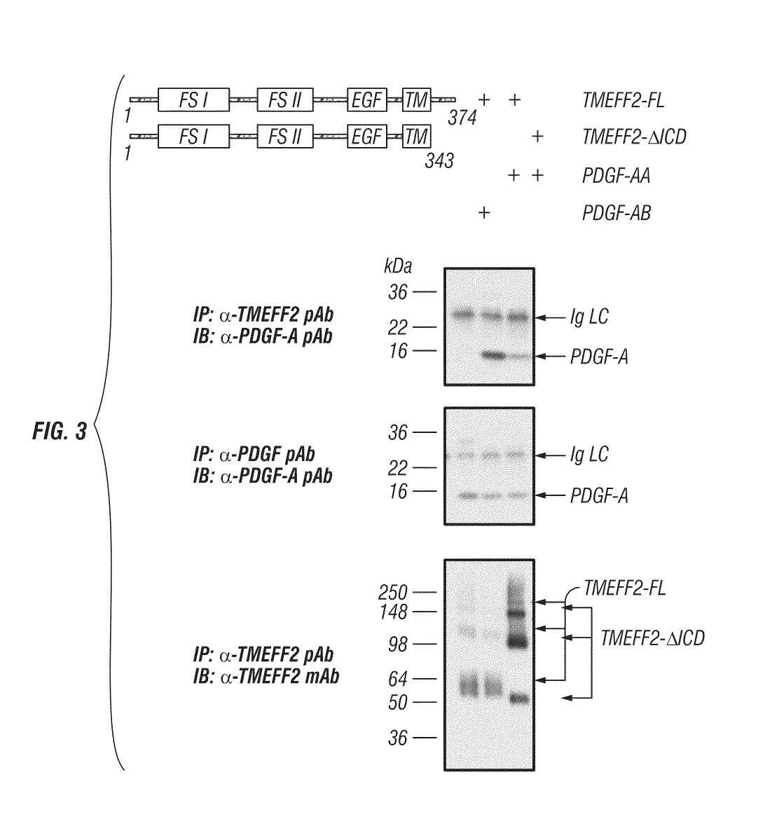

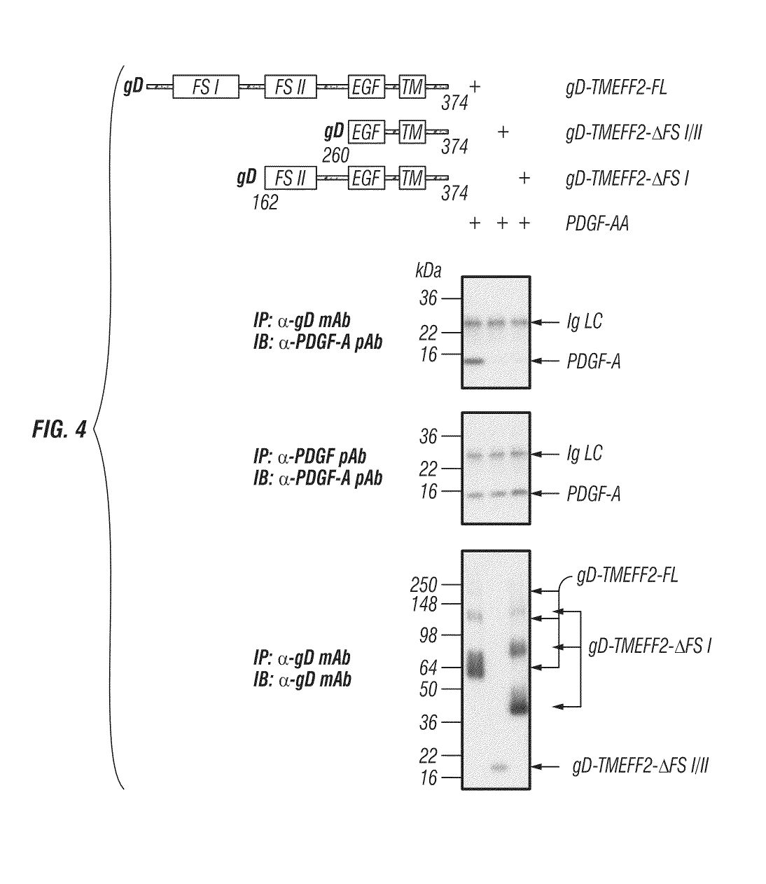

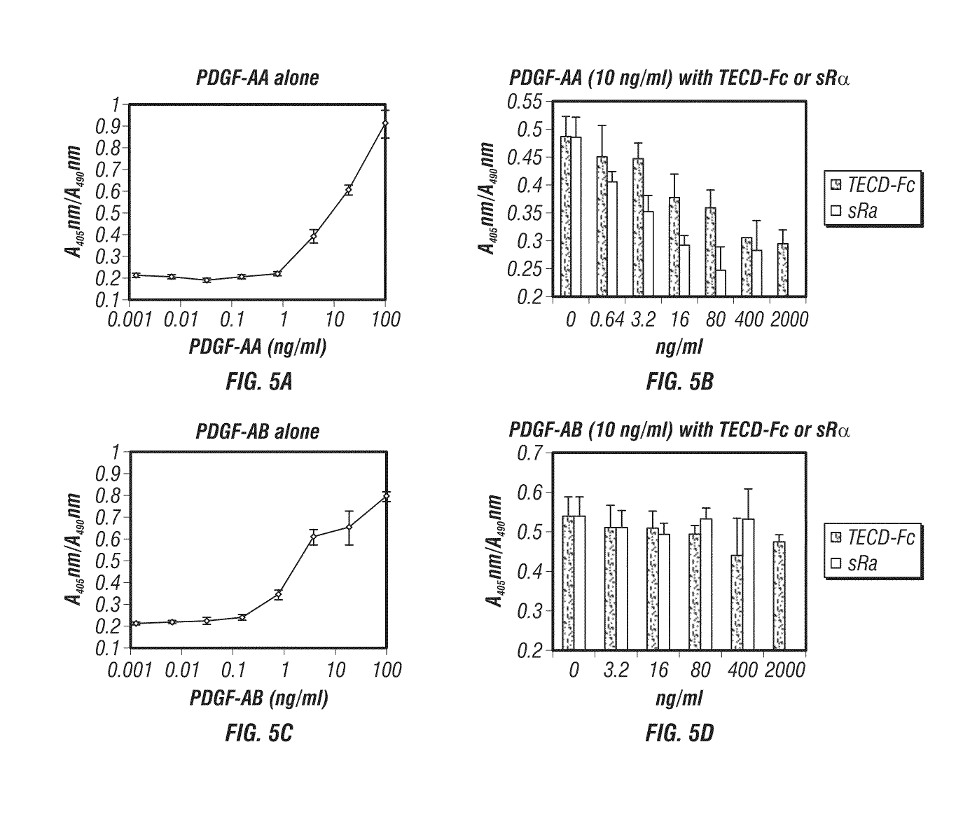

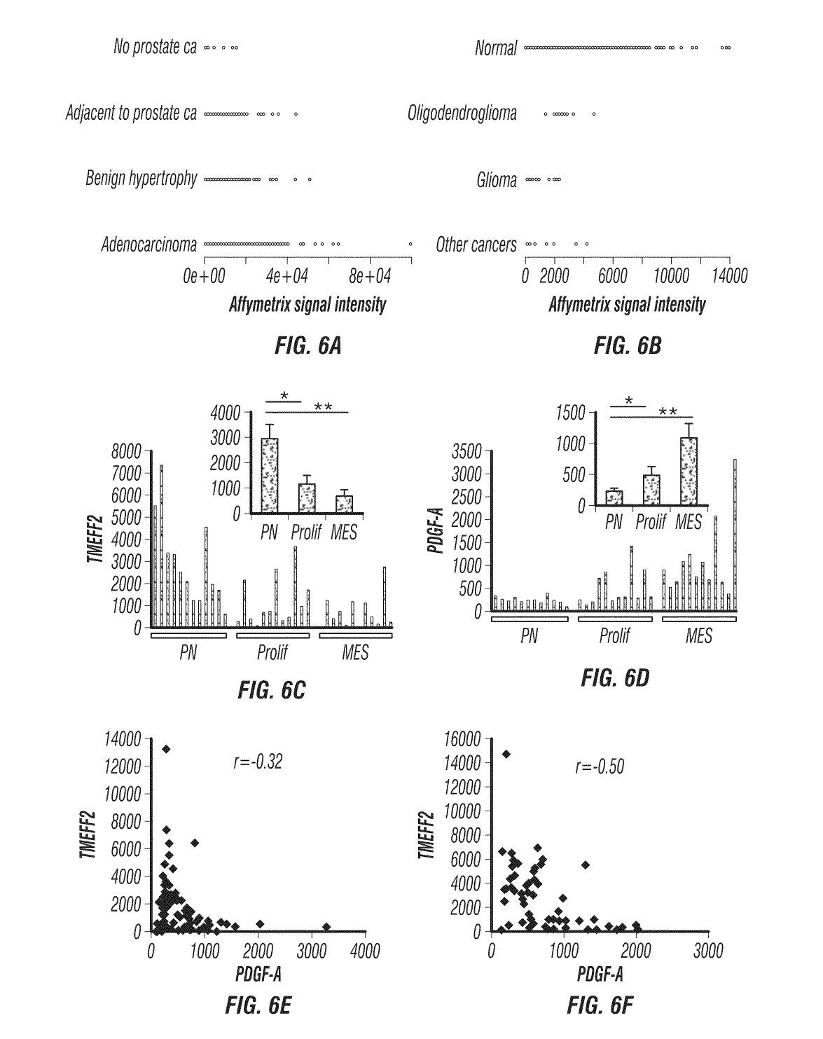

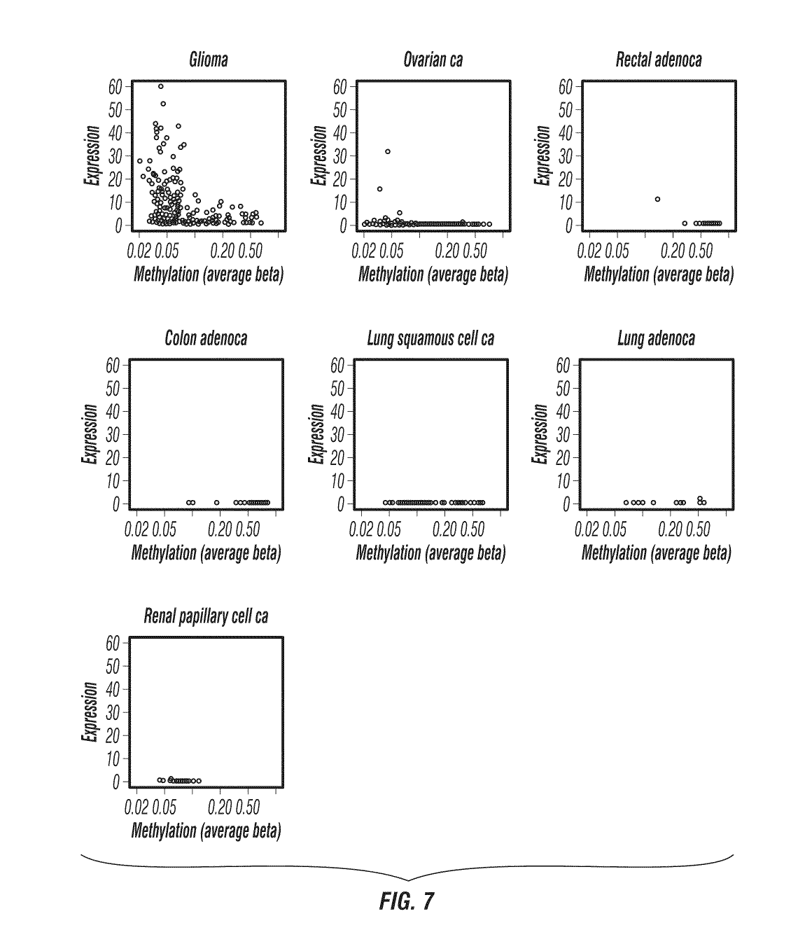

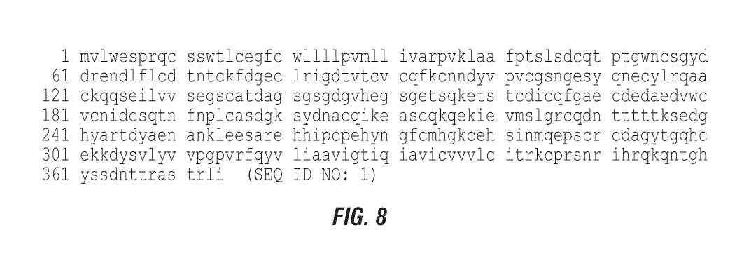

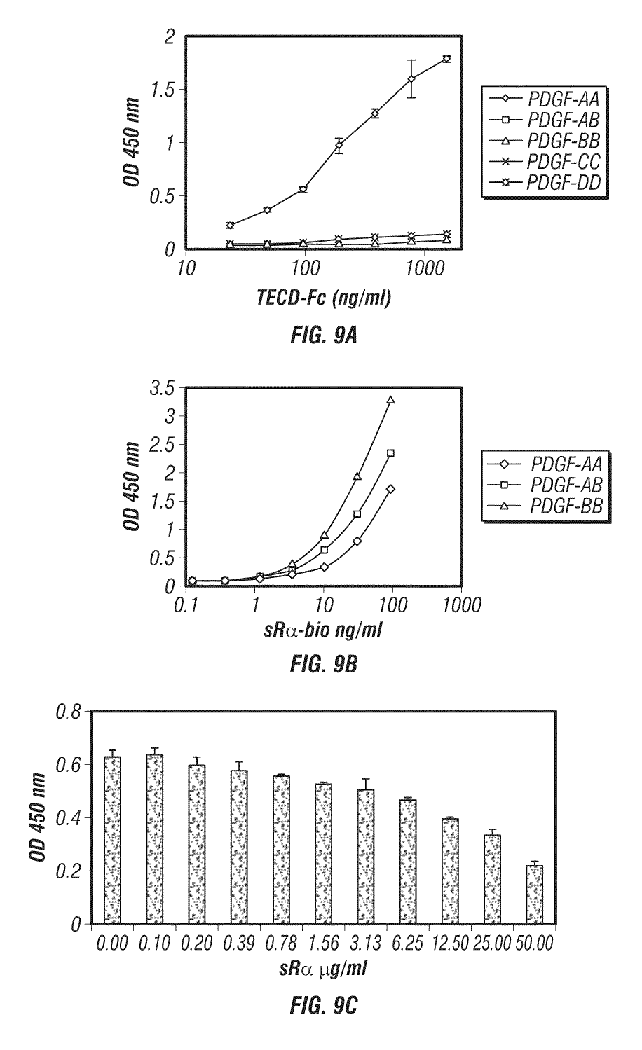

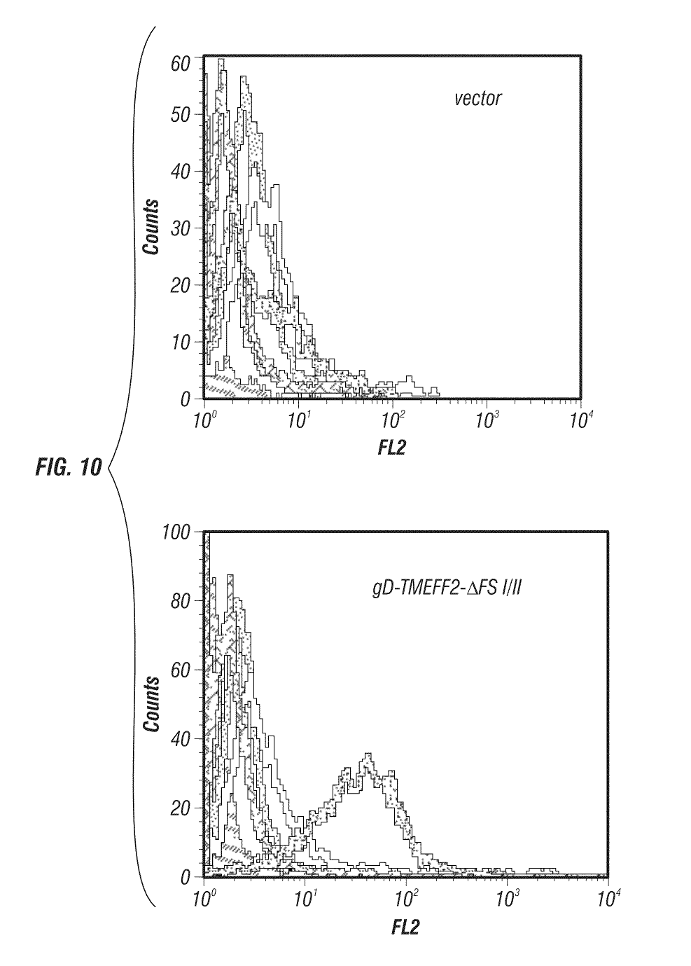

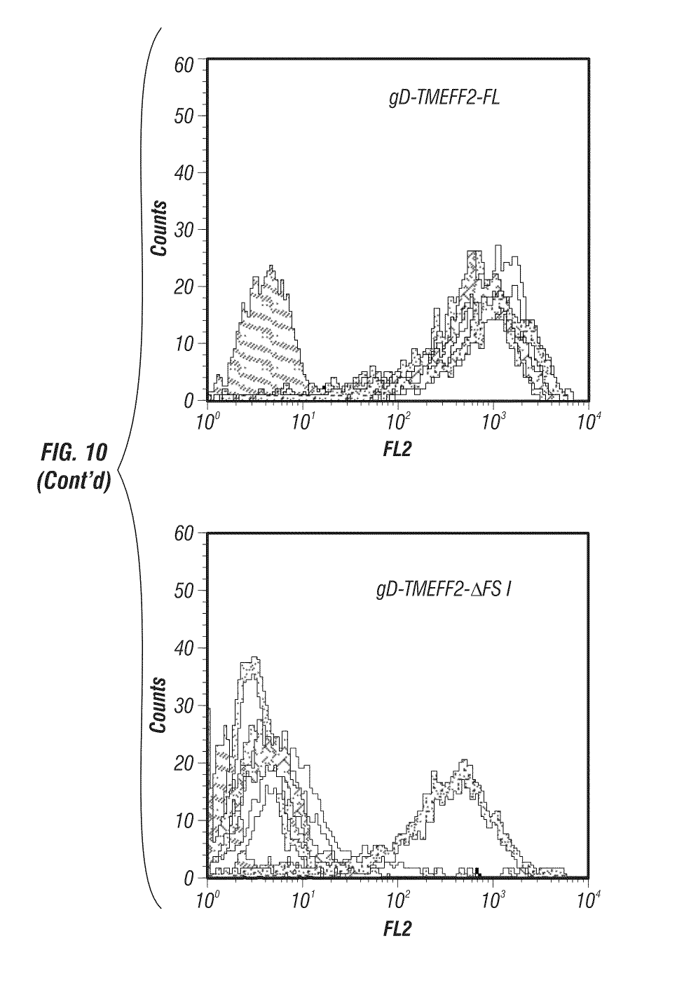

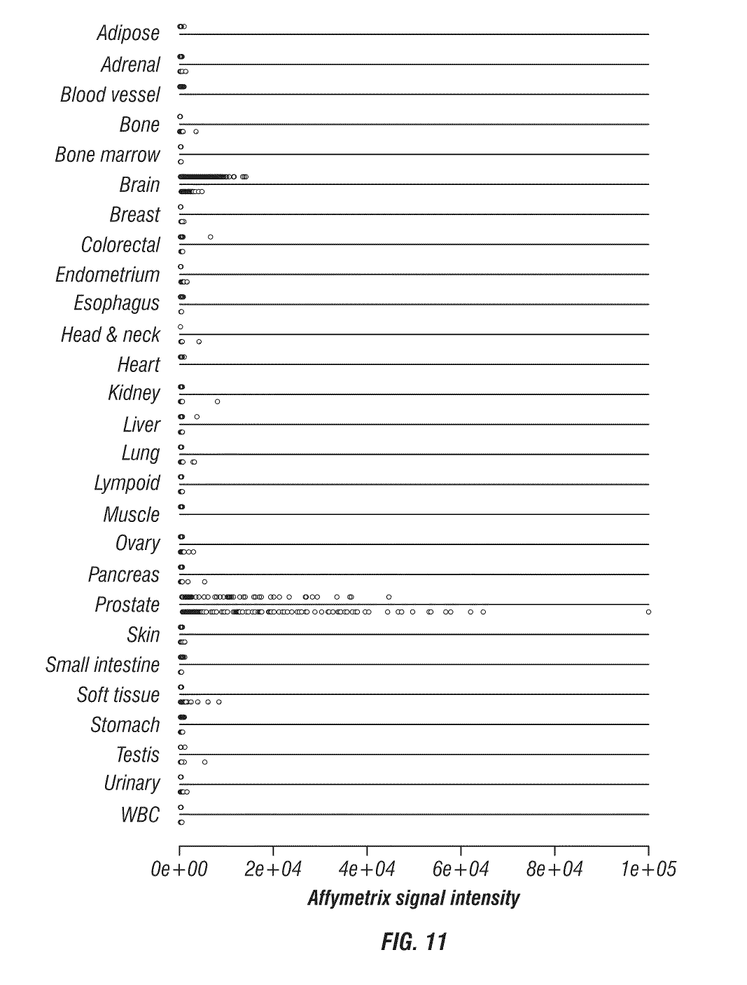

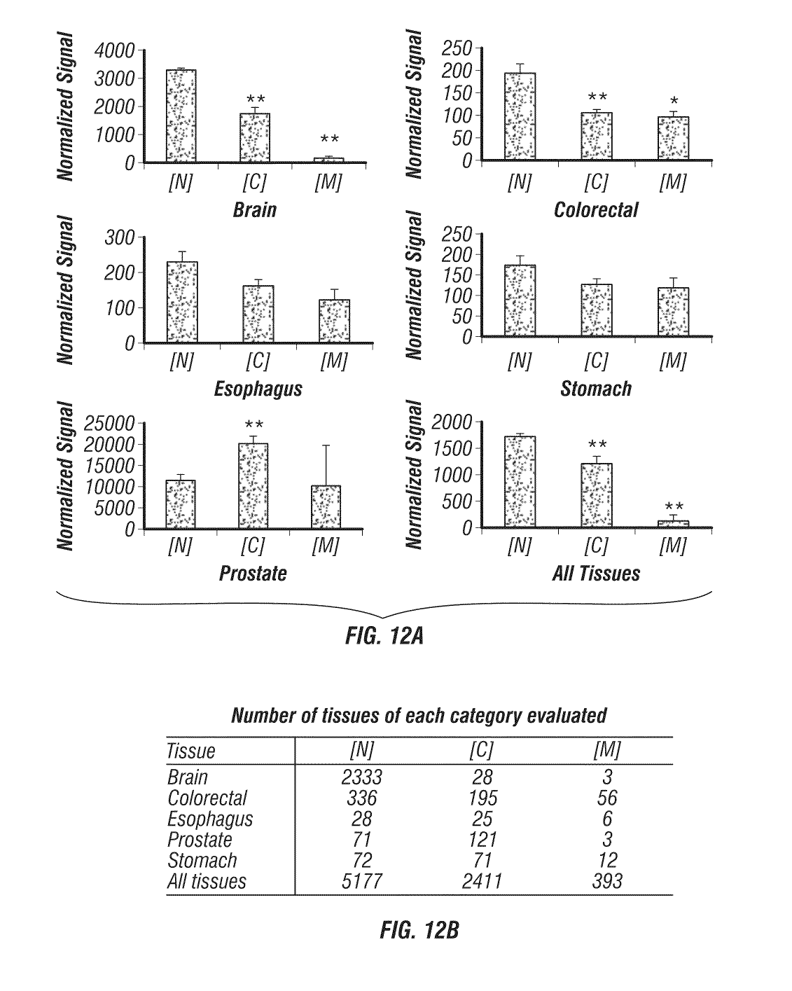

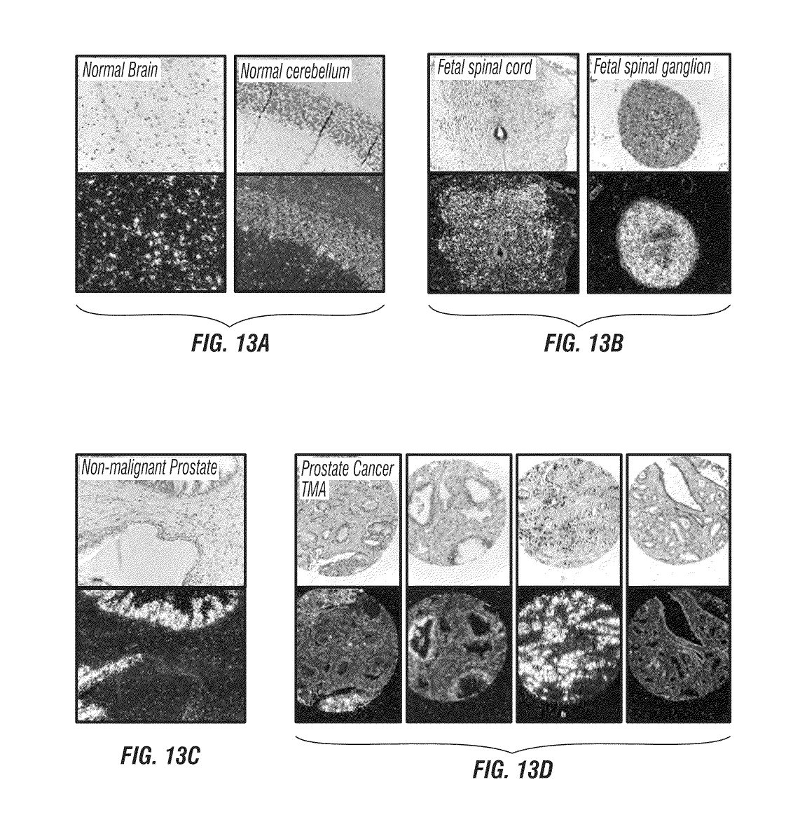

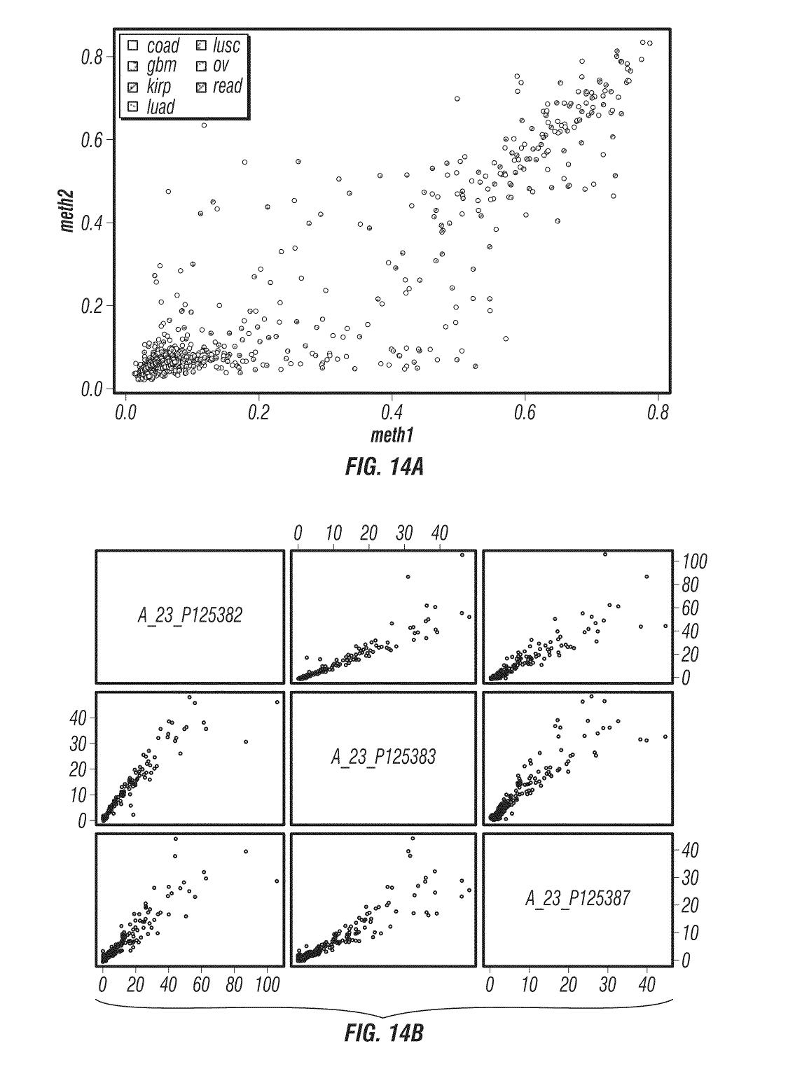

The full-length TMEFF2 open reading frame (GenBank Accession No. NM 016192) was cloned into a modified pRK vector containing a CMV promoter. The FLAG-tagged TECD was cloned into the same vector by PCR amplification using forward primer 5′-CTATCGATCTATCGATATGGTGCTGTGGGAGT-3′ (SEQ ID NO: 2) and reverse primer 5′-GACTCTAGAGTCACTTGTCATCGTCGTCCTTGTAGTCGGCGCGCCACTTTTTTTCA CAGTGTT-3′ (SEQ ID NO: 3) with the FLAG tag (amino acid sequence WRADYKDDDDK, SEQ ID NO: 4) fused in-frame to the CT of the end of the EGF domain. The TECD-Fc was generated similarly using the same forward primer and reverse primer 5′-CTGGGCGCGCCACTTTTTTTCACAGTGTT-3′ (SEQ ID NO: 5) and cloned into the same vector containing the human Fcγsequence which was fused in-frame 3′ to the end of the EGF domain. The gD-tagged full-length TMEFF2 was cloned into the same vector with a 5′ gD tag (amino acid sequence KYALADASLKMADPNRFRGKDLPVLSGR, SEQ ID NO: 6) attached in-frame to the predicted start of the mature protein (residue 41 as shown in FIG. 1D). gD-TMEFF2-ΔFS I and TMEFF2-ΔFS I/II were PRC amplified with the same reverse primer 5′-CGACTCTAGATTAGATTAACCTCGTGGACGCT-3′ (SEQ ID NO: 7) and either 5′-CTGCTCGAGTGTGATATTTGCCAGTTTGGTG-3′ (SEQ ID NO: 8) or 5′-CTGCTCGAGACACCACATACCTTGTCCGGAAC-3′ (SEQ ID NO: 9) as forward primer, respectively.

ELISA to Measure Binding of TMEFF2 and Other Proteins to PDGF

For the TMEFF2 coat format, MaxiSorp 96-well microwell plates (Thermo Scientific Nunc, Roskilde, Denmark) were coated with 1 μg/ml TECD-FLAG in 50 mM carbonate buffer, pH 9.6, overnight at 4° C. Plates were washed with PBS, pH 7.4 containing 0.05% polysorbate 20 and blocked with 0.5% bovine serum albumin (BSA), 15 parts per million Proclin 300 in phosphate buffered saline (PBS), pH 7.4 for 1 hour at room temperature. Serially diluted PDGF-AA, PDGF-AB or PDGF-BB in PBS containing 0.5% BSA, 0.05% polysorbate 20, and 15 parts per million Proclin 300 were added to the plates. After a 2-hour incubation, PDGF bound to the plates was detected by incubating biotinylated goat anti-human PDGF-AA or PDGF-AB or PDGF-BB in the wells for 1 hour, followed by horseradish peroxidase conjugated streptavidin (GE Healthcare, Piscataway, N.J.) for 30 min. Bound Fc-fusion protein was detected by adding goat anti-hman Fc-HRP (Jackson ImmunoResearch, West Grove, Pa.). After a final wash, the substrate 3,3′,5,5′-tetramethyl benzidine (Kirkegaard & Perry Laboratories) was added to plates and the reaction was stopped by adding 1 M phosphoric acid. Plates were washed with PBS, pH 7.4, containing 0.05% Tween 20, between steps and all incubation steps were performed at room temperature on an orbital shaker. Absorbance was read at 450 nm on a multiskan Ascent reader (Thermo Scientific, Hudson, N.H.). The titration curves were fitted using a four-parameter nonlinear regression curve-fitting program (KaleidaGraph, Synergy software, Reading, Pa.).

For testing binding of Fc-fusion proteins or his-tagged proteins to TECD-Flag coated on the plate, horseradish peroxidase conjugated goat anti-human Fc (Jackson ImmunoResearch, West Grove, Pa.) or biotinylated anti-His (Penta-His, Qiagen, Valencia, Calif.) followed by horseradish peroxidase conjugated streptavidin was used for detection. For testing TECD-Flag coat, serially diluted mouse anti-Flag (Sigma, St. Louis, Mo.) was added to the plates followed by horseradish peroxidase conjugated goat anti-mouse Fc (Jackson ImmunoResearch). Serially diluted PDGF-AA was also added to wells coated with goat anti-PDGF for comparison to wells coated with 1 μg/ml TECD-Flag. Bound PDGF-AA was detected as described above.

For the PDGF coat format, plates were coated with 1 μg/ml PDGF-AA, PDGF-AB, PDGF-BB, PDGF-CC, or PDGF-DD. Serially diluted TECD-Fc was added to the plates. Bound TECD-Fc was detected using horseradish peroxidase conjugated goat anti-human Fc (Jackson ImmunoResearch, West Grove, Pa.).

ELISA to Measure Binding of PDGF Receptor a to PDGF

To test binding of soluble PDGF receptor a to PDGF, human recombinant PDGF receptor α extracellular domain (PDGF sRα) was biotinylated using biotin-X-NHS (Research Organics, Cleveland, Ohio). Serially diluted biotinylated human recombinant PDGF sRα was added to PDGF-AA, PDGF-AB or PDGF-BB coated wells. Bound receptor was detected using horseradish peroxidase conjugated streptavidin.

To test blocking of PDGF-AA binding to PDGF sRα by TECD-Fc, serially diluted TECD-Fc was pre-mixed with biotinylated PDGF sRα (final concentration 10 ng/ml) and added to PDGF-AA coated wells. Bound receptor was detected using horseradish peroxidase conjugated streptavidin.

Immunoprecipitation and Western Blot

For binding of PDGF ligands to membrane-bound TMEFF2 proteins, 293 cells were transfected with the various TMEFF2 constructs and changed to fresh growth medium containing 5 μg/ml PDGF-AA or AB 48 hours after transfection. After 1 hour of incubation unbound PDGF ligands were washed away with ice cold PBS and cells were lysed in lysis buffer containing 50 mM Tris pH 8.0, 150 mM NaCl, 1 mM EDTA, 1% NP-40, and protease and phosphatase inhibitors, pre-cleared with protein G SEPHAROSE, and immunoprecipitated with anti-TMEFF2, anti-PDGF, or anti-gD antibodies. The immune complexes were dissociated with SDS sample buffer with (reducing condition) or without (non-reducing condition) β-mercaptoethanol and resolved by 4-20% Tris-Glycine SDS PAGE, transferred to nitrocellulose membranes, and detected with the indicated antibodies using enhanced chemiluminescence.

NR6 Proliferation Assays

The NR6 proliferation assay was carried out using a 5-Bromo-2′-deoxy-uridine (BrdU) labeling and detection kit (Roche). The indicated concentrations of PDGF-AA or AB were added to quiescent confluent cultures of NR6 cells in RPMI 1640 supplemented with 1×Serum Replacement 1 (Sigma) on 96-well microplates, either alone or after pre-mixing with increasing concentrations of TECD-Fc or sRα for 1 hour at 37° C. After 18 hours at 37° C. and 5% CO₂, 10 μl BrdU labeling solution was added to each well and the subsequent labeling and detection were carried out following the manufacturer's protocols. BrdU incorporation was measured as absorbance at 405 nm with a reference wavelength at 490 nm.

Microarray Analysis

Gene expression profiling and analysis of microarray data were performed as previously reported (Phillips et al., 2006) using probe 223557 s at for TMEFF2 and 205463 s at for PDGF-A, respectively.

Methylation and expression analysis of TCGA data

We obtained data from the Cancer Genome Atlas (TCGA) that was publicly available as of Jul. 29, 2010, on both the Illumina Infinium methylation microarray and the Agilent G4502A expression microarray. We correlated samples based on the MAGE tables provided and found dual methylation and expression measurements for 86 colon adenocarcinomas, 226 glioma samples, 36 renal papillary cell carcinomas, 21 lung adenocarcinomas, 69 lung squamous cell carcinomas, 535 ovarian carcinomas, and 53 rectal adenocarcinomas.

Methylation was measured using the beta value taken from the Level 2 files provided by TCGA. From the TCGA array description files, we identified two CpG site methylation probes for TMEFF2: cg06856528 and cg18221862. These probe sequences are located at (−204 to −155) and (−29 to +20) relative to the translation start codon, within a CpG island described previously (Young et al., 2001). We found correlations between the beta values for these probes to be above 0.80 for the colon adenocarcinoma, lung adenocarcinoma, and lung squamous cell carcinoma data sets, but 0.73 for gliomas, 0.67 for rectal adenocarcinomas, 0.62 for ovarian carcinomas, and 0.25 for renal papillary cell carcinomas (FIG. 14A). We used the average of the two beta values as our estimate for methylation levels.

Expression was measured using the antilog of the log2 lowest normalized values from the Level 2 files provided by TCGA. The array description files showed three probes belonging to TMEFF2: A_{—}23 ^{—}P125382, A_{—}23 P_{—}125383, and A_{—}23_P125387. Pairwise correlations among these expression values were 0.94-0.95 (FIG. 14B). We used the average of these probe values as our estimate for expression levels.

Results

The Extracellular Domain of TMEFF2 Interacts with PDGF-AA

TMEFF2 is predicted to contain a transmembrane (TM) domain with an amino terminal (NT) signal peptide sequence (SP) (FIG. 1A). Because the extracellular domain (ECD) of TMEFF2 contains an EGF-like and two follistatin (FS)—like modules, structures known to be involved in protein-protein interactions, Enzyme-Linked Immunosorbent Assays (ELISA) was used to screen a panel of candidate interacting partners. Recombinant proteins containing the ECD of TMEFF2 fused to a FLAG tag (TECD-FLAG) or the Fc portion of the human immunoglobulin gamma (hFcγ (TECD-Fc) at the carboxy-terminus (CT) were expressed in mammalian cells and purified from cell culture supernatants (FIG. 1B). The purified TECD-FLAG and TECD-hFc ran at the predicted ˜55 kDa and ˜70 kDa on SDS PAGE under reducing condition, respectively (FIG. 1C). NT sequencing of the purified proteins revealed that the signal peptide was cleaved between residues 40 and 41 in both recombinant proteins (FIG. 1D).

The purified TECD-FLAG protein was used to coat 96-well plates as a bait, candidate recombinant proteins were used as analytes, and binding was detected using antibodies against either the epitope tags or the candidate proteins themselves. No specific binding was detected for the majority of proteins examined, including the tumor necrosis factor receptor (TNFR) and the EGF Receptor family members (EGFR, HER2, HER3 or HER4) fused to hFcγ. In addition, no significant binding was detected between the TMEFF2 ECDs themselves when TECD-Fc was used as an analyte. As a positive control, an anti-FLAG monoclonal antibody showed dose-dependent binding to the TECD-FLAG coated wells FIG. 2A).

Since FS module-containing proteins have been shown to interact with PDGF ligands

(Raines et al., 1992), we examined the ability of each of the 3 dimeric forms of PDGF ligands, PDGF-AA, BB and AB, to interact with TECD-FLAG. Using a biotinylated anti PDGF-A antibody, we observed a dose-dependent binding when 1 to 10 ng/ml of PDGF-AA was added to the immobilized TECD-FLAG. A weak binding was detected using PDGF-BB and a biotinylated anti PDGF-B antibody, whereas no significant binding was detected for PDGF-AB using the biotinylated anti PDGF-A antibody (FIG. 2B). The binding of PDGF-AA to immobilized TECD-FLAG was comparable to its binding to an immobilized anti PDGF antibody under the same condition, whereas no appreciable binding was detected between any of the PDGF ligands and the uncoated plastic wells.

To confirm that the binding observed is indeed due to the interaction between PDGF-AA and TMEFF2-ECD, we then immobilized the PDGF ligands on the plates, and applied the TMEFF2-ECD fused to a different tag, TECD-Fc, as an analyte. Consistent with the results obtained with immobilized TECD-FLAG, TECD-Fc exhibited significant dose—dependent binding only to immortalized PDGF-AA, but not AB, BB, CC or DD (FIG. 2C; FIG. 9A). A recombinant soluble PDGF receptor α extracellular domain (sRα), on the other hand, showed dose-dependent binding to all 3 immobilized PDGF dimers AA, AB and BB (FIG. 9B), whereas the PDGF receptor β ECD-Fc (PDGFR(β-Fc) fusion protein was not able to bind PDGF-AA (FIG. 2D), consistent with the reported specificity of these receptors (Claesson-Welsh et al., 1989; Seifert et al., 1993; Westermark et al., 1989).

Several other Fc- or His-tagged proteins tested also showed no binding to PDGF-AA coated plates, including another follistatin domain—containing protein FLRG (FIG. 2D, data not shown). In addition, His-tagged tandem array of the EGF domains from TMEFF2 also failed to show any appreciable binding to immobilized PDGF-AA, suggesting that the EGF domain alone can not account for the interaction between the TMEFF2 ECD and PDGF-AA (data not shown).

The Extracellular Regions Containing the Follistatin Modules of TMEFF2 are Important for its Interaction with PDGF-AA

To determine if the ECD of TMEFF2 can interact with PDGF-AA when it is expressed in the context of mammalian cells, 293 cells were transfected with constructs containing the full-length TMEFF2 (TMEFF2-FL), or a truncated TMEFF2 without the intracellular domain (TMEFF2-ΔICD) (FIG. 3). PDGF-AA or PDGF-AB was then added to the culture media and allowed to bind to the cell surface for 30 minutes. Unbound PDGF ligands were subsequently washed away and cell lysates were subjected to immunoprecipitation with either a polyclonal antibody (pAb) recognizing both PDGF-AA and AB dimers, or a pAb recognizing the extracellular domain of TMEFF2. As shown in FIG. 3, an anti PDGF-A antibody could detect the denatured PDGF-A monomer in the anti PDGF immunoprecipitates from cells incubated with either PDGF-AA or PDGF-AB, suggesting that both PDGF dimers bound to the cell surface, either through interactions with specific receptors or extracellular matrix proteins. However, PDGF-A was only detected in the anti TMEFF2 immunoprecipitates from cells incubated with PDGF-AA but not PDGF-AB. In addition, PDGF-AA was present in anti TMEFF2 immunoprecipitates from cells expressing either the full-length TMEFF2 or the ICD-truncated TMEFF2. This is consistent with the ELISA result that PDGF-AA but not PDGF-AB exhibited dose-dependent binding to the ECD of TMEFF2.

TMEFF2 contains 2 FS modules and an EGF-like domain. To dissect which domain(s) of TMEFF2 is/are involved in its interaction with PDGF-AA, Herpes simplex type 1 glycoprotein D (gD)-epitope tagged deletion mutants of TMEFF2 were made and examined for their ability to bind PDGF-AA when expressed on the surface of 293 cells (FIG. 4). As expected, PDGF-AA co-immunoprecipitated with gD-tagged full-length TMEFF2 by an anti gD monoclonal antibody. However, when gD-tagged TMEFF2 mutants lacking either the NT FS I (gD-TMEFF2-ΔFS I) or both of the FS modules (gD-TMEFF2-ΔFS I/II) were immunoprecipitated with the same anti-gD antibody, no PDGF-AA was brought down, although both mutant TMEFF2 proteins were brought down in the immunoprecipitates (FIG. 4). FACS analysis also confirmed membrane expression of all 3 gD-tagged proteins (FIG. 10). This suggests that NT regions containing the FS I domain are required for the PDGF-AA interaction, wherease EGF domain alone is insufficient for this interaction. Specifically, regions amino-terminus to the EGF domain of TMEFF2, including the 2 follistatin domains, 2 N-linked glycosylation sites, and a glycosaminoglycan (GAG) attachment site, are identified as important for the PDGF-AA interaction, whereas EGF domain alone is insufficient for this interaction. The latter is consistent with the ELISA result where the His-tagged tandem array of the TMEFF2 EGF domains failed to bind PDGF-AA (data not shown).

TMEFF2 Modulates PDGF-Stimulated Proliferation of NR6 Fibroblasts

PDGF ligands are potent mitogens of connective tissue cells, including fibroblasts, smooth muscle cells, chondrocytes, and some endothelial cells (Heldin and Westermark, 1999; Heldin and Westermark, 1996; Raines, 1990). The finding that TMEFF2 interacts with PDGF-AA at ng/ml concentrations of both recombinant TMEFF2 extracellular domain (TECD) and PDGF-AA prompted us to examine the possibility that TMEFF2 may regulate PDGF-AA signaling. We first asked whether TECD could compete with PDGFRα, the only receptor that binds PDGF-AA, for PDGF-AA binding. As shown in FIG. 9C, 10 ng/ml of recombinant soluble PDGFRα extracellular domain (sRα) was added to PDGF-AA coated wells in the presence of increasing concentrations of TECD-Fc. Dose-dependent inhibition of sRα—PDGF-AA binding was observed with 4 to 3000 ng/ml of TECD-Fc.

We next examined the effect of TMEFF2-ECD on PDGF stimulated proliferation. The murine fibroblast cell line NR6 cells express both PDGF receptors α and β, and exhibit dose-dependent proliferation in response to PDGF-AA or PDGF-AB as measured by BrdU incorporation (FIG. 5A, C). When 10 ng/ml PDGF-AA was added in the presence of increasing concentrations of Fc-tagged TECD, BrdU incorporation was inhibited in a dose-dependent manner at concentrations between 0.6 and 2,000 ng/ml of TECD-Fc (FIG. 5B). This effect was similar to that of sRα which also inhibited PDGF-AA—induced BrdU incorporation at a similar concentration range, albeit with higher efficiency. PDGF-AB—induced BrdU incorporation, on the other hand, was not affected by TECD-Fc under the same condition (FIG. 5D). Interestingly, sRα also had little effect on PDGF-AB—induced proliferation, although PDGF-AB could bind sRα with an affinity similar to PDGF-AA (FIG. 10B), consistent with previous reports (Claesson-Welsh et al., 1988). PDGF-AB can bind to all 3 PDGFR dimers, αα, αβ, or ββ (Seifert et al., 1993) whereas PDGF-AA can only signal through PDGFR αα dimers. It is possible that PDGF-AB may have a higher affinity for PDGF receptor αβ dimers than for sRα, or that there may be more abundant PDGF receptor aβ dimers and/or PDGF receptor ββ dimers in these cells.

TMEFF2 Expression is Downregulated in Brain Cancers and is Negatively Correlated with PDGF-AA Expression

The 5′-region of TMEFF2 gene is frequently hypermethylated in some cancers (Liang et al., 2000) (Young et al., 2001) (Shibata et al., 2002) (Sato et al., 2002) (Wynter et al., 2004) (Belshaw et al., 2004) (Takahashi et al., 2004) (Geddert et al., 2004) (Suzuki et al., 2005a) (Suzuki et al., 2005b), suggesting a possible tumor suppressor role of TMEFF2 in these cancers. Hypermethylation of TMEFF2 gene is most frequently reported in colorectal and gastric cancers (Liang et al., 2000) (Young et al., 2001) (Shibata et al., 2002) (Sato et al., 2002) (Wynter et al., 2004) (Belshaw et al., 2004) (Geddert et al., 2004). To compare the expression levels of TMEFF2 in human tissues, we analyzed Affymetrix microarray data obtained from Gene Logic, Inc. containing multiple human tumor and normal biopsy samples. Highest levels of TMEFF2 expression were found in prostate and brain tissues (FIGS. 11 and 12). In situ hybridization experiments confirmed high levels of TMEFF2 mRNA expression in normal adult and fetal central nervous systems, as well as both malignant and non-malignant prostate tissues (FIG. 13). The mean expression level of TMEFF2 is significantly higher in prostate cancer tissues compared to normal prostate (FIGS. 11 and 12), consistent with previous reports (Afar et al., 2004). However, in multiple other tissues, especially in brain and colorectal tissues, TMEFF2 exhibits lower mean levels of expression in cancerous and metastatic samples when compared with normal tissues (FIGS. 11 and 12), consistent with a possible tumor suppressor role of TMEFF2 in these tissues.

High grade gliomas (HGGs) can be classified into three molecular subtypes based on similarity to defined expression signatures: proneural (PN), proliferative (Prolif) and mesenchymal (MES) (Phillips et al., 2006). The proneural subtype is distinguished by markedly better prognosis and expresses genes associated with normal brain and the process of neurogenesis. In contrast, the other two subtypes are of poor prognosis characterized by a resemblance to either highly proliferative cell lines or tissues of mesenchymal origin, showing activation of gene expression programs indicative of cell proliferation or angiogenesis, respectively. Microarray analysis of TMEFF2 in 36 samples from newly diagnosed cases of WHO grade III and IV astrocytomas from M.D. Anderson Cancer Center (MDA) revealed significantly higher levels of TMEFF2 expression in the PN subclass than the more malignant Prolif and MES subclasses (FIG. 6C). Interestingly, PDGF-A showed an almost mirror-imaged opposite trend with the highest expression in the MES subclass (FIG. 6D). Such trend was not observed for PDGF-B in these samples (data not shown). Further analysis of microarray data in 76 HGG samples from M.D. Anderson Cancer Center (MDA) suggests a negative correlation between TMEFF2 and PDGF-A expression in both sets of samples (FIG. 6E, F). These data are consistent with the hypothesis that TMEFF2 expression is selected against in HGGs that are dependent on PDGF-AA signaling.

TMEFF2 is Hypermethylated in Multiple Tumor Types with its Expression Negatively Correlated with Methylation Levels.

Hypermethylation of the TMEFF2 gene in human cancers has been reported in several tissues including colorectal, gastric and esophageal cancers (Liang et al., 2000, Young et al., 2001, Belshaw et al., 2004, Geddert et al., 2004, Sato et al., 2002, Shibata et al., 2002, Wynter et al., 2004). However, these tissues express very low levels of TMEFF2 even in normal samples, making the significance of gene suppression less clear in these tumors. Since the methylation status of TMEFF2 has not been reported in glioma and most other tissues, we analyzed all publicly available data from The Cancer Genome Atlas (TCGA) with results on both Agilent expression and Infinium methylation arrays (Bibikova et al., 2009). Of the seven tumor types where these data are currently available, only glioma, and occasionally ovarian and rectal cancer samples show significant levels of TMEFF2 expression (FIG. 7). All samples with high levels of TMEFF2 expression correspond to low CpG island methylation states, while samples with a methylation beta value of greater than 0.1 have a suppressed expression of TMEFF2. Almost all colon adenocarcinoma, rectal adenocarcinoma, lung adenocarcinoma and lung squamous cell carcinoma samples show methylation beta values greater than 0.05, and TMEFF2 expression is barely detectable in all these tumor samples (FIG. 7). These data are consistent with TMEFF2 being silenced through DNA methylation in a significant proportion of human cancers, including glioma and cancers of ovarian, rectal, colon and lung origins.

Discussion

Follistatin domain—containing proteins have been shown to interact with growth factors or their binding partners and modulate their signaling (Harms and Chang, 2003; Phillips and de Kretser, 1998; Raines et al., 1992). For example, the follistatin domain—containing extracellular matrix (ECM)—associated glycoprotein SPARC/osteonectin was reported to interact with PDGF-AB and BB (but not AA) and inhibit the binding of these ligands to their cognate receptors on fibroblasts (Raines et al., 1992). Here we report for the first time that TMEFF2 preferentially interacts with PDGF-AA over PDGF-BB and PDGF-AB via its follistatin domain—containing extracellular regions, and modulates PDGF-AA—stimulated proliferation of NR6 fibroblasts. Interestingly, both shedding of the extracellular domains of TMEFF2 (Lin et al., 2003), and a truncated splice variant of TMEFF2 encoding a secreted protein without the EGF-like and the transmembrane domains (Quayle and Sadar, 2006), have been identified in cancer cells, suggesting a possible functional role of the extracellular region containing the follistatin domains in the ECM.

First identified in a search for serum factors that stimulate the proliferation of arterial smooth muscle cells (Ross et al., 1974), PDGFs have been shown to direct a variety of cellular responses including proliferation, survival, migration, and the deposition of ECM and tissue remodeling factors (reviewed in (Heldin and Westermark, 1999) and (Hoch and Soriano, 2003)). Of the genes encoding the four PDGF ligands and their two receptor chains, mouse knockout studies have suggested that PDGF-B and PDGFRβ are essential for the development of support cells in the vasculature, whereas PDGF-A and PDGFRα are more broadly required during embryogenesis, with essential roles in central nervous system, neural crest and organ development (reviewed in (Hoch and Soriano, 2003)). PDGFs have also been implicated in the etiology of human cancer. Both PDGFs and PDGFRs are upregulated in human gliomas and astrocytomas, and Pdgfra expression levels are higher in more advanced forms of gliomas than in less malignant glial tumors (Hermanson et al., 1992; Hermanson et al., 1996). Elevated levels of PDGF-A and PDGFRα proteins have also been observed in human prostate carcinomas (Fudge et al., 1996; Fudge et al., 1994; Heldin and Westermark, 1999). In human gastric cancers, high levels of PDGF-A correlate with high-grade carcinomas and reduced patient survival (Katano et al., 1998). Pdgfra-activating mutations have also been identified in a subset of human gastrointestinal stromal tumors (Heinrich et al., 2003). Interestingly, we and others have observed highest levels of TMEFF2 expression in the central nervous system and the prostate amongst normal human tissues (FIGS. 11-13 and (Afar et al., 2004)). Conversely, lower levels of TMEFF2 are found in multiple cancer tissues, especially in the malignant brain and colorectal samples, when compared to normal tissues. These findings are consistent with the reported hypermethylation of the TMEFF2 gene in human cancers including colorectal, gastric and esophageal cancers (Liang et al., 2000) (Young et al., 2001) (Shibata et al., 2002) (Sato et al., 2002) (Wynter et al., 2004) (Belshaw et al., 2004) (Geddert et al., 2004) and suggest a possible tumor suppressor role of TMEFF2 in these tissues. In contrast, the mean TMEFF2 mRNA expression is elevated in prostate cancer tissues compared to normal prostates, suggesting a possible tissue and cell context-dependent dual function of TMEFF2 in human cancers. Our findings not only suggest a possible connection between the role of TMEFF2 in PDGF signaling and the potential tumor suppressor function of TMEFF2, but also provide possible explanations for the seemingly conflicting roles of TMEFF2 in human cancers. It is conceivable that the EGF-like domain might have growth factor-like functions opposite to its follistatin domains. For example it was previously reported that soluble forms of TMEFF2 extracellular domain could weakly stimulate erbB-4 tyrosine phosphorylation in MKN 28 gastric cancer cells (Uchida et al., 1999), and promote survival of mesencephalic dopaminergic neurons in primary culture (Horie et al., 2000). Alternatively, the interaction between TMEFF2 and PDGF-AA may either function to sequester the active PDGF ligand away from its receptor, or act as a carrier to concentrate or stabilize the PDGF ligand, depending on the local concentrations of these proteins under different cellular contexts.

### REFERENCES

Afar, D. E. H., V. Bhaskar, E. Ibsen, D. Breinberg, S. M. Henshall, J. G. Kench, M. Drobnjak, R. Powers, M. Wong, F. Evangelista, C. O'Hara, D. Powers, R. B. DuBridge, I. Caras, R. Winter, T. Andersonl, N. Solvason, P. D. Stricker, C. Cordon-Cardo, H. I. Scher, J. J. Grygiel, R. L. Sutherland, R. Murray, V. Ramakrishnan, and D. A. Law. 2004. Preclinical validation of anti-TMEFF2-auristatin E—conjugated antibodies in the treatment of prostate cancer. Molecular Cancer Therapeutics. 3:921-932.
Belshaw, N. J., G. O. Elliott, E. A. Williams, D. M. Bradburn, S. J. Mills, J. C. Mathers, and I. T. Johnson. 2004. Use of DNA from Human Stools to Detect Aberrant CpG Island Methylation of Genes Implicated in Colorectal Cancer. Cancer Epidemiology Biomarkers & Prevention. 13:1495-1501.
Bibikova, M., Le, J., Barnes, B., Saedinia-Melnyk, S., Zhou, L., Shen, R., and Gunderson, K. L. (2009) Epigenomics 1, 177-200
Chang, C., B. J. Eggen, D. C. Weinstein, and A. H. Brivanlou. 2003. Regulation of nodal and BMP signaling by tomoregulin-1 (X7365) through novel mechanisms. Dev Biol. 255:1-11.
Claesson-Welsh, L., A. Eriksson, A. Moren, L. Severinsson, B. Ek, A. Ostman, C. Betsholtz, and C. H. Heldin. 1988. cDNA cloning and expression of a human platelet-derived growth factor (PDGF) receptor specific for B-chain-containing PDGF molecules. Mol Cell Biol. 8:3476-86.
Claesson-Welsh, L., A. Eriksson, B. Westermark, and C. H. Heldin. 1989. cDNA cloning and expression of the human A-type platelet-derived growth factor (PDGF) receptor establishes structural similarity to the B-type PDGF receptor. Proc Natl Acad Sci USA. 86:4917-21.
Fudge, K., D. G. Bostwick, and M. E. Stearns. 1996. Platelet-derived growth factor A and B chains and the alpha and beta receptors in prostatic intraepithelial neoplasia. Prostate. 29:282-6.
Fudge, K., C. Y. Wang, and M. E. Stearns. 1994. Immunohistochemistry analysis of platelet-derived growth factor A and B chains and platelet-derived growth factor alpha and beta receptor expression in benign prostatic hyperplasias and Gleason-graded human prostate adenocarcinomas. Mod Pathol. 7:549-54.
Geddert, H., S. Kiel, E. Iskender, A. R. Florl, T. Krieg, S. Vossen, H. E. Gabbert, and M. Sarbia. 2004. Correlation of hMLH1 and HPP1 hypermethylation in gastric, but not in esophageal and cardiac adenocarcinoma. International Journal of Cancer. 110:208-211. Gery, S., C. L. Sawyers, D. B. Agus, J. W. Said, and H. P. Koeffler. 2002. TMEFF2 is an androgen-regulated gene exhibiting antiproliferative effects in prostate cancer cells. Oncogene. 21:4739-4746.
Glynne-Jones, E., M. E. Harper, L. T. Seery, R. James, I. Anglin, H. E. Morgan, K. M. Taylor, J. M. Gee, and R. I. Nicholson. 2001. TENB2, a proteoglycan identified in prostate cancer that is associated with disease progression and androgen independence. International Journal of Cancer. 94:178-184.
Harms, P. W., and C. Chang. 2003. Tomoregulin-1 (TMEFF1) inhibits nodal signaling through direct binding to the nodal coreceptor Cripto. Genes Dev. 17:2624-9. Epub 2003 Oct. 16.
Heinrich, M. C., C. L. Corless, A. Duensing, L. McGreevey, C. J. Chen, N. Joseph, S. Singer, D. J. Griffith, A. Haley, A. Town, G. D. Demetri, C. D. Fletcher, and J. A. Fletcher. 2003. PDGFRA activating mutations in gastrointestinal stromal tumors. Science. 299:708-10. Epub 2003 Jan 9.
Heldin, C.-H., and B. Westermark. 1999. Mechanism of Action and In Vivo Role of Platelet-Derived Growth Factor. Physiol. Rev. 79:1283-1316.
Heldin, C. H., and B. Westermark. 1996. Role of Platelete-Derived Growth Factor In Vivo. Plenum, NEW YORK. 249-273 pp.
Hermanson, M., K. Funa, M. Hartman, L. Claesson-Welsh, C. H. Heldin, B. Westermark, and M. Nister. 1992. Platelet-derived growth factor and its receptors in human glioma tissue: expression of messenger RNA and protein suggests the presence of autocrine and paracrine loops. Cancer Res. 52:3213-9.
Hermanson, M., K. Funa, J. Koopmann, D. Maintz, A. Waha, B. Westermark, C. H. Heldin, O. D. Wiestler, D. N. Louis, A. von Deimling, and M. Nister. 1996. Association of loss of heterozygosity on chromosome 17p with high platelet-derived growth factor alpha receptor expression in human malignant gliomas. Cancer Res. 56:164-71.
Hoch, R. V., and P. Soriano. 2003. Roles of PDGF in animal development. Development. 130:4769-4784.
Horie, M., Y. Mitsumoto, H. Kyushiki, N. Kanemoto, A. Watanabe, Y. Taniguchi, N. Nishino, T. Okamoto, M. Kondo, T. Mori, K. Noguchi, Y. Nakamura, E.-i. Takahashi, and A. Tanigami. 2000. Identification and Characterization of TMEFF2, a Novel Survival Factor for Hippocampal and Mesencephalic Neurons. Genomics. 67:146-152.
Katano, M., M. Nakamura, K. Fujimoto, K. Miyazaki, and T. Morisaki. 1998. Prognostic value of platelet-derived growth factor-A (PDGF-A) in gastric carcinoma. Ann Surg. 227:365-71.
Kupprion, C., K. Motamed, and E. H. Sage. 1998. SPARC (BM-40, osteonectin) inhibits the mitogenic effect of vascular endothelial growth factor on microvascular endothelial cells. J Biol. Chem. 273:29635-40.
Kyte, J., and R. F. Doolittle. 1982. A simple method for displaying the hydropathic character of a protein. J. Mol. Biol. 157:105-132.
Liang, G., K. D. Robertson, C. Talmadge, J. Sumegi, and P. A. Jones. 2000. The gene for a novel transmemberane protein containing epidermal growth factor and follistatin domains is frequently hypermethylated in human tumor cells. Cancer Research. 60:4907-4912.
Lin, H., K. Wada, M. Yonezawa, K. Shinoki, T. Akamatsu, T. Tsukui, and C. Sakamoto. 2003. Tomoregulin ectodomain shedding by proinflammatory cytokines Life Sci. 73:1617-27.
Mohler, J. L., T. L. Morris, O. H. F. III, R. F. Alvey, C. Sakamoto, and C. W. Gregory. 2002. Identification of differentially expressed genes associated with androgen-independent growth of prostate cancer. The Prostate. 51:247-255.
Patel, K. 1998. Follistatin. Int J Biochem Cell Biol. 30:1087-93.
Patthy, L., and K. Nikolics. 1993. Functions of agrin and agrin-related proteins. Trends Neurosci. 16:76-81.
Phillips, D. J., and D. M. de Kretser. 1998. Follistatin: a multifunctional regulatory protein. Front Neuroendocrinol. 19:287-322.
Phillips, H. S., S. Kharbanda, R. Chen, W. F. Forrest, R. H. Soriano, T. D. Wu, A. Misra, J. M. Nigro, H. Colman, L. Soroceanu, P. M. Williams, Z. Modrusan, B. G. Feuerstein, and K. Aldape. 2006. Molecular subclasses of high-grade glioma predict prognosis, delineate a pattern of disease progression, and resemble stages in neurogenesis. Cancer Cell. 9:157-73.
Polakis, P. 2005. Arming antibodies for cancer therapy. Curr Opin Pharmacol. 5:382-7.
Quayle, S., and M. Sadar. 2006. A truncated isoform of TMEFF2 encodes a secreted protein in prostate cancer cells. Genomics. 87:633-637.
Raines, E. W., Bowen-Pope, D. F., and Ross R. 1990. Platelet-Derived Growth Factor. Springer-Verlag, Heidelberg. 173-262 pp.
Raines, E. W., T. F. Lane, M. L. Iruela-Arispe, R. Ross, and E. H. Sage. 1992. The extracellular glycoprotein SPARC interacts with platelet-derived growth factor (PDGF)-AB and -BB and inhibits the binding of PDGF to its receptors. Proc Natl Acad Sci USA. 89:1281-5.
Ross, R., J. Glomset, B. Kariya, and L. Harker. 1974. A platelet-dependent serum factor that stimulates the proliferation of arterial smooth muscle cells in vitro. Proc Natl Acad Sci USA. 71:1207-10.
Sato, F., D. M. Shibata, N. Harpaz, Y. Xu, J. Yin, Y. Mori, S. Wang, A. Olaru, E. Deacu, F. M. Selaru, M. C. Kimos, P. Hytiroglou, J. Young, B. Leggett, A. F. Gazdar, S. Toyooka, J. M. Abraham, and S. J. Meltzer. 2002. Aberrant Methylation of the HPP1 Gene in Ulcerative Colitis-associated Colorectal Carcinoma. Cancer Research. 62:6820-6822.
Seifert, R. A., A. van Koppen, and D. F. Bowen-Pope. 1993. PDGF-AB requires PDGF receptor alpha-subunits for high-affinity, but not for low-affinity, binding and signal transduction. J Biol. Chem. 268:4473-80.
Shibata, D. M., F. Sato, Y. Mori, K. Perry, J. Yin, S. Wang, Y. Xu, A. Olaru, F. Selaru, K. Spring, J. Young, J. M. Abraham, and S. J. Meltzer. 2002. Hypermethylation of HPP1 Is Associated with hMLH1 Hypermethylation in Gastric Adenocarcinomas. Cancer Research. 62:5637-5640.
Suzuki, M., H. Shigematsu, D. S. Shames, N. Sunaga, T. Takahashi, N. Shivapurkar, T. Iizasa, E. P. Frenkel, J. D. Minna, T. Fujisawa, and A. F. Gazdar. 2005a. DNA methylation-associated inactivation of TGF beta-related genes DRM/Gremlin, RUNX3, and HPP1 in human cancers. British Journal of Cancer. 93:1029-1037.
Suzuki, M., S. Toyooka, N. Shivapurkar, H. Shigematsu, K. Miyajima, T. Takahashi, V. Stastny, A. L. Zern, T. Fujisawa, H. I. Pass, M. Carbone, and A. F. Gazdar. 2005b. Aberrant methylation profile of human malignant mesotheliomas and its relationship to SV40 infection. Oncogene. 24:1302-8.
Takahashi, T., N. Shivapurkar, E. Riquelme, H. Shigematsu, J. Reddy, M. Suzuki, K. Miyajima, X. Zhou, B. N. Bekele, A. F. Gazdar, and I. I. Wistuba. 2004. Aberrant Promoter Hypermethylation of Multiple Genes in Gallbladder Carcinoma and Chronic Cholecystitis. Clinical Cancer Research. 10:6126-6133.
Uchida, T., K. Wada, T. Akamatsu, M. Yonezawa, H. Noguchi, A. Mizoguchi, M. Kasuga, and C. Sakamoto. 1999. A novel epidermal growth factor-like molecule containing two follistatin modules stimulates tyrosine phosphorylation of erbB-4 in MKN28 Gastric cancer cells. Biochemical and Biophysical Research Communications. 266:593-602.
Westermark, B., L. Claesson-Welsh, and C. H. Heldin. 1989. Structural and functional aspects of the receptors for platelet-derived growth factor. Prog Growth Factor Res. 1:253-66. Wynter, C. V., M. D. Walsh, T. Higuchi, B. A. Leggett, J. Young, and J. R. Jass. 2004. Methylation patterns define two types of hyperplastic polyp associated with colorectal cancer. Gut. 53:573-80.
Wynter, C. V., Walsh, M. D., Higuchi, T., Leggett, B. A., Young, J., and Jass, J. R. (2004) Gut. 53, 573-580
Young, J., K. G. Biden, L. A. Simms, P. Huggard, R. Karamatic, H. J. Eyre, G. R. Sutherland, N. Herath, M. Barker, G. J. Anderson, D. R. Fitzpatrick, G. A. Ramm, J. R. Jass, and B. A. Leggett. 2001. HPP1: A transmembrane protein-encoding gene commonly methylated in colorectal polyps and cancers. Proceedings of the National Academy of Sciences of the United States of America. 98:265-270.

1. A method for modulating a PDGF-AA-mediated biological response comprising inhibiting the interaction of TMEFF2 with said PDGF-AA in a cell.

2. The method of claim 1 wherein said cell is a tumor cell.

3. The method of claim 2 wherein said biological response is PDGF-AA-mediated stimulation of proliferation, survival or migration of said tumor cell.

4. The method of claim 3 wherein said modulation is inhibition of the proliferation, survival or migration of said tumor cell.

5. The method of claim 4 wherein the tumor is prostate cancer.

6. The method of claim 5 wherein said prostate cancer is characterized by elevated TMEFF2 expression.

7. The method of claim 6 wherein said prostate cancer is additional characterized by elevated expression of PDGF-A or PDGFRα.

8. The method of claim 3 wherein said inhibition is performed by administration of an agent inhibiting the binding of PDGF-AA to TMEFF2.

9. The method of claim 8 wherein said agent binds to PDGF-AA or TMEFF2.

10. The method of claim 8 wherein said agent is selected from the group consisting of antibodies, TMEFF2 variants and peptide and non-peptide small molecules.

11. The method of claim 2 wherein said biological response is PDGF-AA-mediated tumor suppression.

12. The method of claim 11 wherein said modulation is enhancement of PDGF-AA-mediated tumor suppression.

13. The method of claim 12 wherein the tumor is cancer.

14. The method of claim 13 wherein said cancer is glioblastoma, colon cancer or a cancer of the gastrointestinal tract.

15. The method of claim 13 wherein said cancer is characterized by reduced TMEFF2 expression.

16. The method of claim 13 wherein the cancer is characterized by hypermethylation of TMEFF2.

17. The method of claim 13 wherein said inhibition is performed by administration of an agent inhibiting the binding of PDGF-AA to TMEFF2.

18. The method of claim 17 wherein said agent binds to PDGF-AA or TMEFF2.

19. The method of claim 18 wherein said agent is selected from the group consisting of TMEFF2 fragments, TMEFF2 variants, agonist TMEFF2 antibodies, TMEFF2 and peptide and non-peptide mimetics and antagonists of TMEFF2.

20. The method of claim 11 wherein said agent comprises a TMEFF2 extracellular domain (ECD) sequence, or a variant thereof.

21. The method of claim 11 or claim 20 wherein said agent comprises a TMEFF2 EGF-like domain sequence, or a variant thereof.

22. The method of claim 11 or claim 20 wherein said agent comprises a TMEFF2 follistatin-like domain sequence, or a variant thereof.
